(19) United States
(12) Patent Application Publication
(10) Pub. No.: US 2016/0002056 A1
(43) Pub. Date: Jan. 7, 2016

(54) SYNTHETIC ZINC HECTORITE VIA HYDROTHERMAL PREPARATION

(71) Applicants: Mcguire Meaghan CLARK, Nanuet, NY (US); Bull IVOR, Hopewell Junction, NY (US); Johnson Geoffrey MARK, Wappingers Falls, NY (US); BASF SE, Ludwigshafen (DE)

(72) Inventors: Meaghan Clark Mcguire, Nanuet, NY (US); Ivor Bull, Hopewell Junction, NY (US); Geoffrey Mark Johnson, Wappingers Falls, NY (US)

(73) Assignee: BASF SE, LUDWIGSHAFEN (DE)

(21) Appl. No.: 14/768,608

(22) PCT Filed: Mar. 11, 2014
(86) PCT No.: PCT/US2014/023051

§ 371 Date: Aug. 18, 2015

Related U.S. Application Data
(60) Provisional application No. 61/776,228, filed on Mar. 11, 2013.
(60) Provisional application No. 61/776,262, filed on Mar. 11, 2013.

(51) Int. Cl. C01B 33/32; A61K 8/02; A61Q 19/00; C09K 21/02; C09D 7/12; D21H 17/68; C03C 14/00; C09D 11/322; C08K 3/34; C08J 5/18; C03C 8/00; A61K 8/27; C09D 11/037

(52) U.S. Cl. CPC C01B 33/32; A61K 8/27; A61K 8/0254; A61Q 19/00; C09K 21/02; C09D 7/1216; C09D 7/1225; C09D 11/037; C03C 14/004; C09D 11/322; C08K 3/34; C08J 5/18; C03C 8/00; D21H 17/68; A61K 2800/43; A61K 2800/10; C01P 2004/20; C01P 2004/61

(57) ABSTRACT

This invention relates to synthetically derived zinc hectorite platelets, of superior platelet diameter, effect pigments comprising such synthetically derived platelets and methods of forming said substrates. More specifically the disclosure describes an improved hydrothermal synthesis of zinc hectorite suitable as a platelet for interference pigments, barrier and flame retardant applications.

This application claims the benefit of U.S. provisional application Ser. Nos. 61/776,228, 61/776,262 both filed on Mar. 11, 2013 and both incorporated entirely by reference.

### TECHNICAL FIELD

This application is directed to improved methods of generating synthetic zinc hectorite platelets of large diameter via hydrothermal conditions; the hectorite obtainable by said methods and to the hectorite platelet per se of large diameter.

### BACKGROUND ART

Natural hectorite typically has the formula Na_{0.3}(Mg,Li)₃Si₄O₁₀(OH)₂

It is well known to produce hectorite synthetically via hydrothermal processing. For example, U.S. Pat. Nos. 3,954,943 and 3,586,478 teach the synthesis of fluorine containing hectorite by a hydrothermal process.

It is also known to prepare synthetic zinc hectorite hydrothermally via low temperatures and pressures. See for example, Komarneni, S. et al, Clays and Clay Minerals, Vol. 50, No. 3, 299-305, 2002.

However these synthetic methods do not allow for adequate control of particle size, in particular the platelet diameter of hectorite. The natural hectorite platelet and the synthetic hectorite platelet are normally less than 2 microns in diameter.

Accordingly there a pressing need for a synthetic pathway for clays, in particular hectorite which leads directly to a pure hectorite of large diameter flakes (=>2 microns).

### SUMMARY OF THE INVENTION

The present applicants have discovered several methods (a first and a second method) for producing a synthetic zinc hectorite platelet hydrothermally at low temperatures and pressures which meet the above needs. The produced synthetic zinc hectorite platelet is of a diameter which is =>2 microns.

The first method requires the presence of a habit modifier during hydrothermal synthesis of the synthetic zinc hectorite platelet. Habit modifiers are well known in the art for materials such as zeolites and silver halides but not previously known for hectorite.

The second method does not require the presence of a habit modifier but instead the zinc hectorite platelet is formed via a hydrothermal process which hydrothermal process is modified in such a way as to give platelets of a larger diameter (>2 microns) than previously formed by the known hydrothermal methods.

First Method

The first method is directed to a method of preparing a synthetic zinc hectorite platelet of formula (1)

Iₓ(Zn₃₋ₓ,Liₓ)Si₄O₁₀(X)₂ (1)
wherein
I is an interlayer monovalent cation selected from the group consisting of K⁺, Na⁺, Li⁺,
NH₄⁺ and mixtures thereof, preferably Li⁺, Na⁺ and mixtures thereof;
and
X is independently fluoride or hydroxide, preferably hydroxide;
subscript x is a number ranging from >0 to 1 and including 1;
and Zn and Li are greater than 0;

comprising the steps of:
forming a reaction mixture comprising
an I source selected from the group consisting of K⁺, Na⁺, Li⁺, NH₄⁺ and mixtures thereof, preferably Li⁺, Na⁺ and mixtures thereof;
   a silicon source;
   a lithium source;
   a zinc source;
   optionally a fluoride source and/or hydroxide source, preferably hydroxide;
   a habit modifier wherein the habit modifier is a weak organic or inorganic acid, salt or hydrate thereof or a sugar;
   and
   optionally seed crystal of a preformed hectorite crystals,
hydrothermally treating said reaction mixture under basic conditions at a temperature ranging from about 125 to about 250° C., preferably 150° C. to about 225° C.
and
a pressure ranging from about 50 to about 400 psi, preferably about 100 psi to about 220 psi;
and
optionally isolating the formed platelet.

The Second Method
is directed to the preparation of a synthetic hectorite platelet of formula (1) comprising the steps of:
forming a reaction mixture comprising
an I source selected from the group consisting of K⁺, Na⁺, Li⁺, NH₄⁺ and mixtures thereof, preferably Li⁺, Na⁺ and mixtures thereof;
   a silicon source;
   a zinc source;
   a lithium source;
   optionally a fluoride source and/or hydroxide source, preferably hydroxide;
   and
   optionally seed crystal of a preformed hectorite crystal,
hydrothermally treating said aqueous gel, dispersion or solution under basic conditions at a temperature ranging from about 125 to about 250° C., preferably 150° C. to about 225° C.
and
a pressure ranging from about 50 to about 400 psi, preferably about 100 psi to about 220 psi;
and
optionally isolating the formed platelet, wherein the silicon source is colloidal silica.

This application embodies a synthetic zinc hectorite platelet of formula (1) obtainable by the process (first method or second method above) described above.

Furthermore this application embodies a synthetic zinc hectorite of formula (1) wherein the diameter is =>2 microns.

The above zinc hectorite platelet is envisioned as an effect pigment wherein the platelet is coated with at least one metal oxide layer.

The above zinc hectorite platelet is also envisioned as barrier additive in polymers packaging and the like. The hectorite platelet of a diameter=>2 microns is especially useful for this application.

The inventors also claim the use of a habit modifier and a method to increase the diameter of a synthetic zinc hectorite platelet during hydrothermal synthesis of the synthetic platelet, wherein the habit modifier is a weak organic or inorganic acid, salt or hydrate thereof or a sugar.

### DETAILED DESCRIPTION OF THE INVENTION

### Definitions

The term “hydrothermal process” as used herein means a process that allows platelets of material such as synthetic zinc hectorite to grow in a solvent at temperatures and pressures which allow for the at least partial dissolution or dispersion of precursor materials.

The terms “synthetically derived” means the zinc hectorite is formed synthetically, i.e. by a controlled chemical reaction, specifically a hydrothermal reaction. The hydrothermal reaction conditions disclosed herein are those characterized by low temperature and low pressures.

The terms “low temperature” and “low pressure” when used to describe the hydrothermal process conditions means for purposes of this application temperatures ranging from 125 to about 250° C., preferably 150° C. to about 225° C. and pressures ranging from about 50 to about 400 psi, and preferably about 100 psi to about 220 psi.

The term “platelet, platy, plate-like and flakey” are typical terms used in the art and is understood to mean that the platy substrates have a diameter which is greater than the thickness of the substrate, such as platelets (flakes).

The diameter is defined as the d₅₀ particle size distribution determined via static light scattering using a Malvern Mastersizer® Hydo2000S. The thickness of the platelet is determined via Scanning Electron Microsope (SEM).

The reaction mixture will typically be an aqueous dispersion, solution, slurry or gel of the starting materials.

Bulk density is a property of powders or granules and other “divided” materials such as the platy zinc hectorite substrates formed by the process disclosed herein. This bulk density is defined as the weight of a unit volume of the powder usually expressed as grams per cubic centimeter.

This is also sometimes referred to as the apparent density. It accounts for the powder and the voids between particles as well as voids within and on the surface of the particles.

### The Zinc Hectorite Platelet

Hectorite for purposes of this application means a zinc containing hectorite of formula (1)

Iₓ(Zn₃₋ₓ,Liₓ)Si₄O₁₀(X)₂ (1)

I is an interlayer cation which binds and is sandwiched between the tetrahedral units (Zn₃₋ₓ, Li_{z}) and the octahedral units Si₄O₁₀.
I is an interlayer monovalent cation selected from the group consisting of K⁺, Na⁺, NH₄⁺, Li⁺ and mixtures thereof, preferably Li⁺, Na⁺ or mixtures thereof;
The subscript x is a number and ranges from >0 to 1 and including 1,
Zn and Li are greater than 0,
and
X is independently fluoride, hydroxide or a combination of the both fluoride and hydroxide, preferably hydroxide,
The above platelet is formed via use of a habit modifier during hydrothermal processing and the habit modifier is selected from the group consisting of organic weak acids, inorganic weak acids and sugars, or the zinc hectorite platelet without use of a habit modifier but modification of the hydrothermal process by using (silicon source) colloidal silica or a mixture of the two modifications (use of colloidal silica and use of habit modifier).
Typically the synthetic hectorite will correspond to any one of a number of structures such as Liₓ(Zn₃₋ₓLiₓ)Si₄O₁₀(OH)₂, Naₓ(Zn₃₋ₓLiₓ)Si₄O₁₀(OH)₂, Kₓ(Zn₃₋ₓLiₓ)Si₄O₁₀(OH)₂, (NH₄)ₓ(Zn₃₋ₓLiₓ)Si₄O₁₀(OH)₂, Liₓ(Zn₃₋ₓLiₓ)Si₄O₁₀(F)₂, Naₓ(Zn₃₋ₓLiₓ)Si₄O₁₀(F)₂, Kₓ(Zn₃₋ₓLiₓ)Si₄O₁₀(F)₂, (NH₄)ₓ(Zn₃₋ₓLiₓ)Si₄O₁₀(F)₂, Liₓ(Zn₃₋ₓLiₓ)Si₄O₁₀(F,OH), Naₓ(Zn₃₋ₓLiₓ)Si₄O₁₀(F,OH), Kₓ(Zn₃₋ₓLiₓ)Si₄O₁₀(F,OH), (NH₄)ₓ(Zn₃₋ₓLiₓ)Si₄O₁₀(F,OH), (Li,K)ₓ(Zn₃₋ₓLiₓ)Si₄O₁₀(OH)₂, (Li,K)ₓ(Zn₃₋ₓLiₓ)Si₄O₁₀(F)₂, (Li,K)ₓ(Zn₃₋ₓLiₓ)Si₄O₁₀(F,OH), (Li,Na)ₓ(Zn₃₋ₓLiₓ)Si₄O₁₀(OH)₂, (Li,Na)ₓ(Zn₃₋ₓLiₓ)Si₄O₁₀(F)₂, (Li,Na)ₓ(Zn₃₋ₓLiₓ)Si₄O₁₀(F,OH), (Li,K)ₓ(Zn₃₋ₓLiₓ)Si₄O₁₀(OH)₂, (Li, K)ₓ(Zn₃₋ₓLiₓ)Si₄O₁₀(F)₂, (Li, K)ₓ(Zn₃₋ₓLiₓ)Si₄O₁₀(OH, F), (Li,NH₄)ₓ(Zn₃₋ₓLiₓ)Si₄O₁₀(OH)₂, (Li, NH₄)ₓ(Zn₃₋ₓLiₓ)Si₄O₁₀(F)₂, (Li,NH₄)ₓ(Zn₃₋ₓLiₓ)Si₄O₁₀(OH, F).

Additionally, the formation of platelets of a diameter=>2 microns via hydrothermal formation with and without habit modifier is also of particular importance.

### Identification of the Crystal Form of the Hectorite Crystals

The hectorite crystals are identified via PXRD (Powder X-Ray Diffraction) using CuKα radiation source.

### Habit Modifier

The term “habit” when used in reference to a crystalline substance is a well known term in the art. For example the crystalline habit of mica is monoclinic with formation of extremely thin sheets. A modifier of the habit might alter the geometric structure in such a way as to speed the growth of a lattice plane.

Habit modifiers are known for use in modifying the habit of zeolites. For example, Lupulescu A. I, et al, Angew. Chem. Int. Ed. 2012, 51, 3345-3349 and U.S. Publication No. 2012/0202006 teach spermine for tailoring the crystal habits of zeolites.

However, the present application embodies the use of weak organic acids or weak inorganic acids, hydrates or salts thereof or sugars, as habit modifiers during hydrothermal preparation of synthetic zinc hectorite.

The term weak organic acids means for purposes of this application that the weak organic acid (salt or hydrate) thereof comprises at least one carboxylic acid (salt or hydrate), preferably at least two carboxylic acids (salts or hydrates).

The weak organic acid salts or hydrates thereof as habit modifiers may be defined by formula (I)

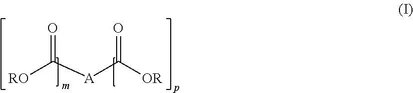

when m+p is 1:

A is branched or unbranched, substituted or unsubstituted C₁-C₁₀ alkyl, branched or unbranched, substituted or unsubstituted C₂-C₁₀ alkenyl, substituted or unsubstituted C₇-C₉ phenylalkyl or substituted or unsubstituted C₆-C₁₀ aryl,

wherein the linear or branched unsubstituted C₁-C₁₀ alkyl, the linear or branched C₂-C₁₀ alkenyl may be substituted by C(O)OH, C(O)O⁻X⁺, NH₂, halogen, OH, —C(O)H or interrupted by —O—, —NR²— or —C(O)—,
the C₇-C₉ phenylalkyl or the C₆-C₁₀ aryl includes substitution by one or more C(O)OH, C(O)O⁻X⁽⁺⁾ⁿ, NH₂, halogen, OH or —C(O)H,
wherein R is hydrogen or RO is O⁻X⁽⁺⁾ⁿ,
R² is hydrogen or linear or branched C₁-C₁₀-alkyl one or more substituted by C(O)OH, C(O)O⁻X⁽⁺⁾ⁿ, halogen, NH₂, —C(O)— or OH; n is 1-3,
and
X⁽⁺⁾ⁿ is a organic or inorganic cation, for example ammonium, substituted ammonium, such as, for example methylammonium, dimethylammonium, trimethylammonium, ethanolammonium, metal cations, for example metal cations for example alkali metal cation, alkaline earth metal cation or other metal cations, for example Na⁺, Li⁺, K⁺, Cs⁺, Rb⁺, Fr⁺, Mg⁺⁺, Sr⁺⁺, Ba⁺⁺, Be⁺⁺, Ca⁺⁺, P⁺⁺⁺, B⁺⁺⁺ or Al⁺⁺⁺.

when m+p is two or more,

A is branched or unbranched, substituted or unsubstituted C₁-C₁₀ alkylene, branched or unbranched, substituted or unsubstituted C₂-C₁₀ alkylidene, substituted or unsubstituted C₇-C₉ alkylphenylene or C₆-C₁₀ arylene,

wherein C₇-C₉ alkylphenylene or C₆-C₁₂ arylene may include one or more substitution by NHR², OH, COOH, halogen, COO⁻X⁽⁺⁾ⁿ or —C(O)H,
and the linear or branched C₁-C₁₀ alkylene, the linear or branched C₂-C₁₀ alkylidene may be substituted by C(O)OH, C(O)O⁻X⁺, NH₂, halogen, OH, —C(O)H and/or interrupted by —O—, —NR²— or —C(O)—,
with R, RO, R² and X⁽⁺⁾ⁿ as defined above.

Preferably m+p is two or more.

C₁-C₁₀ alkyl having up to 10 carbon atoms is a branched or unbranched radical, for example methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, isoheptyl, 1,1,3,3-tetramethylbutyl, 1-methylheptyl, 3-methylheptyl, n-octyl, 2-ethylhexyl, nonyl and decyl.

C₂-C₁₀ alkenyl having up to 10 carbon contains at least one unsaturated carbon-carbon bond. For example, alkenyl is a version of alkyl, for example isopropenyl, propenyl, hexenyl, heptenyl, and the like.

C₇-C₉phenylalkyl is, for example, benzyl, α-methylbenzyl, α,α-dimethylbenzyl or 2-phenylethyl. For example benzyl and α,α-dimethylbenzyl.

C₆-C₁₀ aryl is for example phenyl or naphthyl, but also comprised are hydroxy, halogen NH₂, C(O)H, COOH, COO—X⁽⁺⁾ⁿ substituted phenyl or naphthyl. For example benzoic acid, phthalic acid and terephtalic acid or halogen substituted benzoic acid.

C₁-C₁₀alkylene is a branched or unbranched radical, for example methylene, ethylene, propylene, trimethylene, tetramethylene, pentamethylene, hexamethylene, heptamethylene, octamethylene, decamethylene, dodecamethylene or octadecamethylene. For example C₁-C₁₂alkylene, for instance preferably C₁-C₈alkylene or C₁-C₆ alkylene.

C₂-C₁₀alkylene interrupted by oxygen, NR² or C(O) is, for example, —CH₂—O—CH₂—, —CH₂—NR²CH₂—, —CH₂—N(CH₃)—CH₂—, —CH₂—O—CH₂CH₂—O—CH₂—, —CH₂—(O—CH₂CH₂—)₂O—CH₂—, —CH₂—(O—CH₂CH₂—)₃O—CH₂—, —CH₂—(O—CH₂CH₂—)₄O—CH₂—, —CH₂CH₂—N(CH₂CH₂OH)—CH₂CH₂—, —CH₂CH₂C(O)CH₂CH₂—.

C₂-C₁₀ alkylidene having from 2 to 20 carbon atoms is, for example, ethylidene, propylidene, butylidene, pentylidene, 4-methylpentylidene, heptylidene, nonylidene, tridecylidene, nonadecylidene, 1-methylethylidene, 1-ethylpropylidene or 1-ethylpentylidene. For example C₂-C₈ alkylidene.

C₇-C₉ alkylphenylene is for example, CH₂-Ph-CH₂ (Ph is phenyl), CH₂—CH₂-Ph-CH₂—.
C₆-C₁₂ arylene is for example

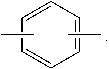

X⁽⁺⁾ⁿ wherein n is 1, 2 or 3. Thus X⁽⁺⁾ⁿ is mono-, di- or tri-valent metal or organic cation.
X⁽⁺⁾ⁿ is for example ammonium, substituted ammonium, such as, for example methylammonium, dimethylammonium, trimethylammonium, ethanolammonium, metal cations, for example metal cations for example alkali metal cation, alkaline earth metal cation or other metal cations, for example Na⁺, Li⁺, K⁺, Cs⁺, Rb⁺, Fr+, Mg⁺⁺, Sr⁺⁺, Ba⁺⁺, Be⁺⁺, Ca⁺⁺, B⁺⁺⁺, P⁺⁺⁺ or Al⁺⁺⁺.
Preferred for formula (I) salt or hydrates thereof are

when m+p is two or more,

A is branched or unbranched, substituted or unsubstituted C₁-C₈ alkylene,

substitution of the branched or unbranched C₁-C₈ alkylene includes one or more substitution by OH, COOH, COO⁻X⁽⁺⁾ⁿ as defined above, preferably OH and COOH, COO⁻X⁽⁺⁾ⁿ substitution.

Suitable weak organic acid habit modifiers would include formic acid, acetic acid, acrylic acid, benzoic acid, phthalic acid, isothalic acid, terephthalic acid, malonic acid, methyl malonic acid, succinic acid, lactic acid, sorbic acid, ascorbic acid, aspartic acid, glutaric acid, adipic acid, pimelic acid, oxalic acid, malic acid, maleic acid, tartaric acid, tartonic acid, mucic acid, gluconic acid, citric acid, isocitric acid, acetyl citric acid, suberic acid, sebacic acid, azelaic acid, 1,2,3-propanetricarboxylic acid, 1,1,3,3-propanetetracarboxylic acid, 1,1,2,2-ethane tetracarboxylic acid, 1,2,3,4-butantetetracarboxylic acid, 1,2,2,3 propanetetracarboxylic acid, 1,3,3,5 pentanetetracarboxylic acid, ethylenediamine tetraacetic acid, ethyleneglycolbis-tetraacetic acid, diglycolic acid, ethylenediamine tetrapropionic acid, iminodiacetic acid, 1,2-propylenediaminetetraacetic acid, N-methyl, -ethyl, -propyl and -butyl iminodiacetic acid, 1,3-propylenediaminetetraacetic acid, N-hydroxyethylethylenediaminetriacetic acid, triethylenetetraminehexaacetic acid, diethylenetriaminepentaacetic acid, amino acids such as glycine, alanine, valine, leucine, tyrosine, thoreonine, serine, glutamic acid, lysine, and salts or hydrates thereof.

A preferred listing of suitable weak organic acids is malic acid, adipic acid, tartaric acid, oxalic acid, tartronic acid, citric acid, isocitric acid, pimilic acid, azelaic acid, dilycolic acid, mucic acid, malonic acid, methyl malonic acid, glutaric acid, succinic acid, aspartic acid, suberic acid, sebacic acid, glutamic acid and salts or hydrates thereof.

A most preferred listing of suitable weak organic acids is malic acid, oxalic acid, tartaric acid, citric acid, isocitric acid, mucic acid and salts or hydrates thereof.

Weak inorganic acids are for example boric acid, phosphoric acid (H₃PO₄), pyrophosphates triphosphates, salts or hydrates thereof.

Sugars are also envisioned as habit modifiers including mono and disaccharides. For example sugars would include glucose, fructose, galactose, sucrose, maltose, sorbitol, lactose mannitol, inositol, xylitol, threitol, erythritol, adonitol(ribitol), arabitol(lyxitol), dulcitol(galactitol), maltitol, isomalt, ribose, xylose and mannose.

The most preferred habit modifiers are weak organic acids or weak inorganic acids such as are citric and boric acid salts and hydrates thereof. For example tri-sodium citrate dehydrate, disodium tartrate dihydrate and tetraborate decahydrate come to mind.

The amount of habit modifier (sugars or weak organic or weak inorganic acids) required during the hydrothermal processing of the zinc hectorite of formula (I) will range from about 0.5 to about 10% mmol, preferably about 1 to about 7% mmol, and most preferably about 1.5 to about 5% mmol based on the theoretical calculated product (hectorite).

### Formation of a Synthetic Zinc Hectorite Under Hydrothermal Conditions

With Habit Modifier

The first method is directed to a method of preparing a synthetic zinc hectorite platelet of formula (1)

Iₓ(Zn₃₋ₓ,Liₓ)Si₄O₁₀(X)₂ (1)
wherein
I is an interlayer monovalent cation selected from the group consisting of K⁺, Na⁺, Li⁺, NH₄⁺ and mixtures thereof, preferably Li⁺, Na⁺ and mixtures thereof;
and
X is independently fluoride or hydroxide or a combination of fluoride or hydroxide, preferably hydroxide;
subscript x is a number ranging from >0 to 1 and including 1;
and Zn and Li are greater than 0;
comprising the steps of:
forming a reaction mixture comprising
an I source selected from the group consisting of K⁺, Na⁺, Li⁺, NH₄⁺ and mixtures thereof, preferably Li⁺, Na⁺ and mixtures thereof;
   a silicon source;
   a lithium source;
   a zinc source;
   optionally a fluoride source and/or hydroxide source, preferably hydroxide;
   a habit modifier wherein the habit modifier is a weak organic or inorganic acid, salt or hydrate thereof or a sugar;
   and
   optionally seed crystal of a preformed hectorite crystals,
hydrothermally treating said reaction mixture under basic conditions at a temperature ranging from about 125 to about 250° C., preferably 150° C. to about 225° C.
and
a pressure ranging from about 50 to about 400 psi, preferably about 100 psi to about 220 psi;
and
optionally isolating the formed platelet.

Without Habit Modifier

The zinc hectorite platelet of formula (1) may also be formed using the second hydrothermal process, that is using colloidal silica as the silicon source.

The second method:
is directed to the preparation of a synthetic hectorite platelet of formula (1) comprising the steps of:
forming a reaction mixture comprising
an I source selected from the group consisting of K⁺, Na⁺, Li⁺, NH₄⁺ and mixtures thereof, preferably Li⁺, Na⁺ and mixtures thereof;
   a silicon source;
   a zinc source;
   a lithium source;
   optionally a fluoride source and/or hydroxide source, preferably hydroxide;
   and
   optionally a seed crystal of a preformed hectorite seed crystal,
hydrothermally treating said aqueous gel, dispersion or solution under basic conditions at a temperature ranging from about 125 to about 250° C., preferably 150° C. to about 225° C.
and
a pressure ranging from about 50 to about 400 psi, preferably about 100 psi to about 220 psi;
and
optionally isolating the formed platelet, wherein the silicon source is colloidal silica.

Methods one and two may be combined, that is into a third method of hydrothermal synthesis wherein the silicon source is colloidal silica and a habit modifier is used during the hydrothermal synthesis.

The diameter is for example defined as the d₅₀ particle size distribution determined via static light scattering using a Malvern Mastersizer® Hydo2000S. The thickness of the platelet is determined via cross sectional Scanning Electron Microsope (SEM).

The synthetic zinc hectorite platelet formed from the first, second and third methods above form a platelet of formula (1) which has a diameter of =>2 microns.

The synthetic zinc hectorite platelet is characterized by a d₅₀ ranging from =>2 to about 60 microns, most preferably about 3 to about 50 microns, especially the platelets may be characterized by a d₅₀ of at least 3 microns.

It is presently preferred that the diameter of the hydrothermally prepared zinc hectorite, range from about 2 microns to about 1 mm with a more preferred range of about 2.5 microns to about 60 microns, especially about 3 microns to about 50 microns.

The synthetic zinc hectorite platelet is substantially transparent, that is it transmits at least 92% light, preferably 95% light and most preferably 98% light.

Use of a habit modifier to increase the diameter of a synthetic zinc hectorite platelet of formula (1)

The applicants also claim the use of a habit modifier to increase the diameter of a synthetic zinc hectorite platelet of formula (1) during hydrothermal synthesis and the habit modifier is a weak organic acid, a weak inorganic acid, salts or hydrates thereof or a sugar.

The habit modifier used to increase the diameter of the synthetic zinc hectorite platelet is a weak organic acid, salt or hydrate thereof and is defined by the formula (I)

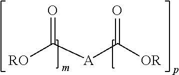

when m+p is 1:
A is branched or unbranched, substituted or unsubstituted C₁-C₁₀ alkyl, branched or unbranched, substituted or unsubstituted C₂-C₁₀ alkenyl, substituted or unsubstituted C₇-C₉ phenylalkyl or substituted or unsubstituted C₆-C₁₀ aryl,
wherein the linear or branched unsubstituted C₁-C₁₀ alkyl, the linear or branched C₂-C₁₀ alkenyl may be substituted by C(O)OH, C(O)O⁻X⁺, NH₂, halogen, OH, —C(O)H or interrupted by —O—, —NR²— or —C(O)—,
the C₇-C₉ phenylalkyl or the C₆-C₁₀ aryl includes substitution by one or more C(O)OH, C(O)O⁻X⁽⁺⁾ⁿ, NH₂, halogen, OH or —C(O)H,
R is hydrogen or RO is O⁻X⁽⁺⁾ⁿ,
R² is hydrogen or linear or branched C₁-C₁₀-alkyl one or more substituted by C(O)OH, C(O)O⁻X⁽⁺⁾ⁿ, halogen, NH₂, —C(O)— or OH; n is 1-3,
and
X⁽⁺⁾n is a organic or inorganic cation, for example ammonium, substituted ammonium, such as, for example methylammonium, dimethylammonium, trimethylammonium, ethanolammonium, metal cations, for example metal cations for example alkali metal cation, alkaline earth metal cation or other metal cations, for example Na⁺, Li⁺, K⁺, Cs⁺, Rb⁺, Fr⁺, Mg⁺⁺, Sr⁺⁺, Ba⁺⁺, Be⁺⁺, Ca⁺⁺, P⁺⁺⁺, B⁺⁺⁺ or Al⁺⁺⁺.
when m+p is two or more,
A is branched or unbranched, substituted or unsubstituted C₁-C₁₀ alkylene, branched or unbranched, substituted or unsubstituted C₂-C₁₀ alkylidene, substituted or unsubstituted C₇-C₉ alkylphenylene or C₆-C₁₀ arylene,
wherein the branched or unbranched C₁-C₁₀ alkylene or branched or unbranched C₂-C₁₀ alkylidene may be substituted by one or more C(O)OH, C(O)O⁻X⁺, NH₂, halogen, OH, —C(O)H and/or interrupted by —O—, —NR²— or —C(O)—,
and
the C₇-C₉ alkylphenylene or the C₆-C₁₂ arylene may be substituted by one or more NHR², OH, COOH, halogen, COO⁻X⁽⁺⁾ⁿ or —C(O)H,
with R, RO, R² and X⁽⁺⁾ⁿ as defined above,
or
the habit modifier is a weak inorganic acid and selected from the group consisting of boric acid, phosphoric acid (H₃PO₄), triphosphates and salts or hydrates thereof.

Alternatively a sugar may be used as the habit modifier to increase the diameter of the zinc hectorite. In this case the habit modifier is a sugar selected from the group consisting of glucose, fructose, galactose, sucrose, maltose, sorbitol, lactose mannitol, inositol, xylitol, threitol, erythritol, adonitol(ribitol), arabitol(lyxitol), dulcitol(galactitol), maltitol, isomalt, ribose, xylose and mannose.

A method of increasing the diameter of a zinc hectorite platelet is envisioned by adding a habit modifier during the hydrothermal preparation and the habit modifier is a weak organic acid, a weak inorganic acid, salts or hydrates thereof or a sugar.

### Particle Size Distribution

A particularly useful means of characterizing the size distribution of a mass of synthetic platelets produced is by specifying the platelet size of the lowest 10 vol. %, 50 vol. %, and 90 vol. % of platelets along the Gaussian curve. This classification can be characterized as the d₁₀, d₅₀, and d₉₀ values of the platelet size distribution. Thus, a platelet having a d₁₀ of a certain size means that 10 vol. % of the platelet particles has a size up to that value. Thus, the size distribution of the hectorite platelets can be described as follows: 10 volume % of the platelets have a size of up to and including 10 microns, 50 volume % of the platelets have a size up to and including 22 microns, and 90 volume % of the platelets have a size up to and including 45 microns for example.

For example the synthetically derived zinc hectorite platelets is preferably characterized by a d₅₀ ranging from about 2 microns to about 60 microns, most preferably about 3 to about 50 microns, especially the synthetically derived zinc hectoric platelets may be characterized by a d₅₀ of at least 3, 4 or 5 microns.

The platelet may of course be classified by means of various methods, such as gravity sedimentation, sedimentation in a decanter, sieving, use of a cyclone or hydrocylone, spiral classifying or a combination of two or more these methods. A method such as sieving, for example, may also be used in a plurality of successive steps. Classification may shift the distribution of platelet toward larger or smaller diameters.

### Hydrothermal Process Variables

As explained above the term “hydrothermal process” as used herein means a process that allows crystals of zinc hectorite platelet to grow in a solvent at low temperature and low pressure.

The solvent is typically water.

The reaction mixture may be a slurry, gel, dispersion or solution.

Thus the preparation of the synthetic zinc hectorite platelet of formula (1) (method 1),
comprises the steps of:
forming a reaction mixture comprising
an I source selected from the group consisting of K⁺, Na⁺, NH₄⁺ and mixtures thereof, preferably Li⁺, Na⁺ and mixtures thereof;
an lithium source, a silicon source; a zinc source,
   optionally a fluoride source and/or hydroxide source, preferably hydroxide;
   a habit modifier and
   optionally a seed crystals of a preformed zinc hectorite,
hydrothermally treating said reaction mixture at a temperature ranging from
about 150 to about 250° C. and a pressure ranging from about 50 to about 400 psi under basic conditions to form the platelets of the synthetic zinc hectorite;
and
optionally isolating the formed synthetic hectorite.
The preparation of the synthetic zinc hectorite platelet of formula (1) (method 2), comprises the steps of:
forming an reaction mixture comprising
an I source selected from the group consisting of K⁺, Na⁺, Li⁺, NH₄⁺ and mixtures thereof, preferably Li⁺, Na⁺ and mixtures thereof;
an lithium source, a silicon source; a zinc source,
   optionally a fluoride source and/or hydroxide source, preferably hydroxide;
   and
   optionally a seed crystals of a preformed zinc hectorite,
hydrothermally treating said reaction mixture at a temperature ranging from
about 150 to about 250° C. and a pressure ranging from about 50 to about 400 psi under basic conditions to form the platelets of the synthetic zinc hectorite;
and
optionally isolating the formed synthetic hectorite, wherein the silicon source is colloidal silica.

It is also possible to combine the two methods above, that is use colloidal silica as the silicon source and a habit modifier.

The reaction may be carried out in a sealed or unsealed vessel.

The base may for example be derived from common inorganic bases such as potassium hydroxide, sodium hydroxide, lithium hydroxide, sodium carbonate, lithium carbonate, ammonium hydroxide, and potassium carbonate and organic basis such as tripropylammonium hydroxide, tetramethyl ammonium hydroxide, triethanolamine and diethanolamine.

Preferably the base is an inorganic base and is selected from the group consisting of lithium hydroxide, lithium carbonate, and potassium carbonate, preferably lithium hydroxide and lithium carbonate. Note that the base may provide the source for I (interlayer cation) in formula (1).

The initial reaction mixture should be basic. Typically the pH of the initial reaction mixture will range from about 7 to about 14, preferably the pH will range from about 8 to about 14, and most preferably will range for about 9 to about 14.

I as explained above, is an interlayer monovalent cation selected from the group consisting of Na⁺, K⁺, NH₄⁺ and Li⁺ and mixtures thereof. The source for this cation may be from the base used to insure basic reaction conditions of the hydrothermal process. For example, bases which would provide the Na⁺ or Li⁺ may be sodium hydroxide, Na₂O, lithium hydroxide, sodium carbonate, lithium carbonate, Li₂O, potassium carbonate and K₂O.

Preferably the interlayer monovalent cation is Na⁺ or Li⁺ or mixtures thereof,

The silica sources are typically derived from hydrates of SiO₂, colloidal SiO₂, sodium metasilicate, sodium silicate, potassium metasilicate, potassium silicate, kaolin, fumed silica, talc, H₂SiO₃ and tetraethyl orthosilicate.

Colloidal SiO₂ is preferred.

### Lithium Source

The lithium resource may be elemental, any salt (organic or inorganic), hydrate or oxide thereof. For example the lithium source may be lithium acetate, lithium bromide, lithium iodide, lithium chloride, lithium fluoride, lithium carbonate, lithium citrate, lithium formate, lithium hexafluorophosphate, lithium hexafluorotitanate, lithium hydroxide, lithium oxide and hydrates thereof.

### Zinc Source

The zinc source may be elemental, any salt (organic or inorganic), hydrate or oxide thereof. The zinc source may be selected from the group consisting of Zn⁰, ZnSO₄, Zn(NO₃)₂, ZnCl₂, Zn(C₂H₃O₂)₂ (zinc acetate), ZnCO₃, Zn(CHO₂)₂ (zinc formate), ZnBr₂, zinc oxide, ZnI₂and hydrates thereof.

### Fluoride Source

The optional fluoride source is for example HF, NH₄F, NaF, K₂SiF₆, KF and MgF₂.

It is preferable not to include a fluoride source.

The water content during the hydrothermal reaction may vary from about 60 to about 98 wt. percent. Thus the wt. % reagents will normally range from about 2 wt. % to about 40 wt. percent, preferably from about 4 wt. % to about 35 wt. % and most preferably about 5 wt. % to about 30 wt. %. The weight % is based on the total weight of the reaction mixture.

### Hydroxide Source

The hydroxide source may come from the bases such as potassium hydroxide, sodium hydroxide, lithium hydroxide, ammonium hydroxide, and organic bases such as tripropylammonium hydroxide and tetramethyl ammonium hydroxide.

It is preferable to have a hydroxide source in both methods 1 and 2.

Thus the process for making the zinc hectorite of formula (1) comprises the steps of:
forming a reaction mixture comprising
   an I source selected from the group consisting of Na⁺, K⁺, NH₄⁺ and Li⁺,
preferably Na⁺ or Li⁺ or a mixture thereof;
   an aluminum source;
   a silicon source;
   a zinc source;
   optionally a fluoride source and/or an hydroxide source; preferably an hydroxide source with an optional fluoride source, most preferably a hydroxide source;
hydrothermally treating said reaction mixture under basic conditions at a temperature ranging from about 125 to about 250° C. preferably 150° C. to about 225° C.
and
a pressure ranging from about 50 to about 400 psi, preferably about 100 psi to about 220 psi;
to form the synthetic hectorite platelet of formula (1);
and
optionally isolating the formed platelet
and the habit modifier is a weak organic acid, weak inorganic acid or a sugar and/or the silicon source is colloidal silica.

### Seeding

Seeding of the hydrothermal reaction may be desirable with a previously formed hectorite. The amount of seeding making up the reaction mixture may range from about 1 to 6 wt. percent of the calculated hectorite platelet, preferably the calculated hectorite product. For example if the product intended is a hectorite, the hydrothermal reaction may be seeded with a wt. % of hectorite seed crystal ranging from about 0.1 to about 10 wt. %, preferably 0.5 to about 8 wt. %, most preferably 1 to about 6 wt. % of the theoretical product formed.

### Time

The hydrothermal reaction mixture is typically heated to the appropriate temperature, about 150 to about 250° C., then held at the appropriate temperature from about 2 to about 100 hours, more typically about 4 to 90 hours or most typically about 6 to about 85 hours.

The pressure conditions for carrying out the hydrothermal reactions will vary depending upon the platelet but will typically vary from about 50 psi to about 400 psi, more typically about 75 psi to about 300 psi, most typically from about 85 to about 250 psi.

The hydrothermal process for production of the hectorite may be done under static or stirring/mixing conditions.

### Stoichiometry of the Hydrothermal Process

The hydrothermal preparation of the zinc hectorite can be done under stoichiometric conditions or non-stoichiometric conditions. It is preferable that the reaction is run under stoichiometric conditions.

Stoichiometric conditions means for purposes of this application, that the starting materials, in particular zinc source, silicon source, and lithium sources are present at the start of the reaction at the same molar ratios of the final product, the zinc hectorite.

### Applications for Hydrothermally Produced Hectorite

There are many applications for the presently disclosed zinc hectorite. For example, hectorite is an excellent insulator, reinforcement material, solid lubricant, cosmetic extender, substrate or core for effect and interference pigments, barrier in packaging and paper applications and filler in resins providing heat resistance.

### Effect Pigment Using the Synthetic Zinc Hectorite

When the term “synthetic zinc hectorite” is use in the follow passages, what is meant is the inventive synthetic hectorite formed via the herein disclosed hydrothermal method or the hydrothermally produced hectorite disclosed herein. These products give platelet=>2 microns which is likely to lead to special visual effects and improved barrier effects.

The two methods two described above give synthetic hectorite of a diameter equal to or greater than 2, 3, 4 or 5 microns and above which gives improved barrier effects and makes possible the use of hectorite as a substrate for interference pigments.

Effect pigments and their use in paints, ink-jet printing, for dyeing textiles, for pigmenting coatings, printing inks, plastics, cosmetics, glazes for ceramics and glass is well known in the art.

Such pigments having a core consisting of a transparent carrier material, such as, for example, natural, or synthetic mica, SiO₂, or glass, are known. Reference is made, for example, to Gerhard Pfaff and Peter Reynders, Chem. Rev. 99 (1999) 1963-1981.

The presently formed substrate, the synthetically derived zinc hectorite, may be an especially suitable substrate, core or platelet for formation of an effect pigment.

One of the objects of the present invention is to develop pearlescent pigments on the basis of the presently hydrothermally produced zinc hectorite with the disclosed platelet diameter of 2 microns or greater. The presently coated synthetic zinc hectorite would exhibit the advantages of mica pigments (e.g. good application properties in a variety of binder systems, environmental compatibility and simple handling) with the possibility of realizing superior optical effects, i.e. to provide interference pigments, having high color strength and/or color purity because of the of the synthetic hectorite platelet of a diameter of 2 or greater.

This objective has been solved by pigments, comprising a plate-like substrate of the hydrothermally produced platelets of zinc hectorite of a diameter equal to or greater than 2 microns, or 3 microns,

(a) a dielectric material, especially a metal oxide, having a high index of refraction; and/or
(a) a metal layer, especially a thin semi-transparent metal layer.

The pigment particles (coated core of synthetically produced hectorite) generally have a diameter of from 2, 3, 4, or 5 microns to 5 mm, and an average thickness of <1 micron, and contain a core of synthetically derived hectorite, having two substantially parallel faces, the distance between which is the shortest axis of the core. The core is either coated with a dielectric material, especially a metal oxide, having a high index of refraction, or a metal layer, especially a thin semi-transparent metal layer. Said layers can be coated with additional layers.

Suitable metals for the (semi-transparent) metal layer are, for example, Cr, Ti, Mo, W, Al, Cu, Ag, Au, or Ni. The semi-transparent metal layer has typically a thickness of between 5 and 25 nm, especially between 5 and 15 nm.

According to the present invention the term “aluminum” comprises aluminum and alloys of aluminum. Alloys of aluminum are, for example described in G. Wassermann in Ullmanns Enzyklopadie der Industriellen Chemie, 4. Auflage, Verlag Chemie, Weinheim, Band 7, S. 281 to 292. Especially suitable are the corrosion stable aluminum alloys described on page 10 to 12 of WO00/12634, which comprise besides of aluminum silicon, magnesium, manganese, copper, zinc, nickel, vanadium, lead, antimony, tin, cadmium, bismuth, titanium, chromium and/or iron in amounts of less than 20% by weight, preferably less than 10% by weight.

The metal layer can be obtained by wet chemical coating or by chemical vapor deposition, for example, gas phase deposition of metal carbonyls. The substrate is suspended in an aqueous and/or organic solvent containing medium in the presence of a metal compound and is deposited onto the substrate by addition of a reducing agent. The metal compound is, for example, silver nitrate or nickel acetyl acetonate (WO03/37993).

According to U.S. Pat. No. 3,536,520 nickel chloride can be used as metal compound and hypophosphite can be used as reducing agent. According to EP-A-353544 the following compounds can be used as reducing agents for the wet chemical coating: aldehydes (formaldehyde, acetaldehyde, benzalaldehyde), ketones (acetone), carbonic acids and salts thereof (tartaric acid, ascorbinic acid), reductones (isoascorbinic acid, triosereductone, reductine acid), and reducing sugars (glucose). However, it is also possible to use reducing alcohols (allyl alcohol), polyols and polyphenols, sulfites, hydrogensulfites, dithionites, hypophosphites, hydrazine, boron nitrogen compounds, metal hydrides and complex hydrides of aluminium and boron. The deposition of the metal layer can furthermore be carried out with the aid of a CVD method. Methods of this type are known. Fluidised-bed reactors are preferably employed for this purpose. EP-A-0741170 describes the deposition of aluminium layers by reduction of alkylaluminium compounds using hydrocarbons in a stream of inert gas. The metal layers can furthermore be deposited by gas-phase decomposition of the corresponding metal carbonyls in a heatable fluidised-bed reactor, as described in EP-A-045851. Further details on this method are given in WO93/12182. A further process for the deposition of thin metal layers, which can be used in the present case for the application of the metal layer to the substrate, is the known method for vapour deposition of metals in a high vacuum. It is described in detail in Vakuum-Beschichtung [Vacuum Coating], Volumes 1-5; Editors Frey, Kienel and Löbl, VDI-Verlag, 1995. In the sputtering process, a gas discharge (plasma) is ignited between the support and the coating material, which is in the form of plates (target). The coating material is bombarded with high-energy ions from the plasma, for example argon ions, and thus removed or atomised. The atoms or molecules of the atomised coating material are precipitated on the support and form the desired thin layer. The sputtering process is described in Vakuum-Beschichtung [Vacuum Coating], Volumes 1-5; Editors Frey, Kienel and Löbl, VDI-Verlag, 1995. For use in outdoor applications, in particular in the application in vehicle paints, the pigments can be provided with an additional weather-stabilising protective layer, the so-called post-coating, which simultaneously effects optimum adaptation to the binder system. Post-coatings of this type have been described, for example, in EP-A-0268918 and EP-A-0632109.

If pigments with metallic appearance are desired, the thickness of the metal layer is >25 nm to 100 nm, preferably 30 to 50 nm. If pigments with colored metal effects are desired, additional layers of colored or colorless metal oxides, metal nitrides, metal sulfides and/or metals can be deposited. These layers are transparent or semi-transparent. It is preferred that layers of high index of refraction and layers of low index of refraction alternate or that one layer is present, wherein within the layer the index of refraction is gradually changing. It is possible for the weathering resistance to be increased by means of an additional coating, which at the same time causes an optimal adaption to the binder system (EP-A-268918 and EP-A-632109).

In one preferred embodiment of the present invention, the interference pigments comprise materials having a “high” index of refraction, which is defined herein as an index of refraction of greater than about 1.65, and optionally materials having a “low” index of refraction, which is defined herein as an index of refraction of about 1.65 or less. Various (dielectric) materials that can be utilized including inorganic materials such as metal oxides, metal suboxides, metal fluorides, metal oxyhalides, metal sulfides, metal chalcogenides, metal nitrides, metal oxynitrides, metal carbides, combinations thereof, and the like, as well as organic dielectric materials. These materials are readily available and easily applied by physical, or chemical vapor deposition processes, or by wet chemical coating processes.

Optionally a SiO₂ layer can be arranged between the inventive hectorite substrate and the materials having a “high” index of refraction. By applying a SiO₂ layer on the substrate the mica surface is protected against chemical alteration, such as, for example, swelling and leaching of mica components. The thickness of the SiO₂ layer is in the range of 5 to 200 nm, especially 40 to 150 nm. The SiO₂ layer is preferably prepared by using an organic silane compound, such as tetraethoxy silane (TEOS). The SiO₂ layer can be replaced by thin layers (thickness 1 to 20 nm) of Al₂O₃, Fe₂O₃ or ZrO₂.

Furthermore, the SiO₂-coated, or TiO₂-coated synthetic mica flakes may, as described in EP-A-0 982 376, be coated with a nitrogen-doped carbon layer. The process described in EP-A-0 982 376 comprises the following steps:

(a) suspending the SiO₂, or TiO₂ coated synthetic mica flakes in a liquid,
(b) where appropriate adding a surface-modifier and/or a polymerization catalyst,
(c), before or after step (b), adding one or more polymers comprising nitrogen and carbon atoms, or one or more monomers capable of forming such polymers,
(d) forming a polymeric coating on the surface of the flakes,
(e) isolating the coated flakes and
(f) heating the coated flakes to a temperature of from 100 to 600° C. in a gaseous atmosphere.

The polymer may be a polypyrrole, a polyamide, a polyaniline, a polyurethane, a nitrile rubber or a melamine-formaldehyde resin, preferably a polyacrylonitrile, or the monomer is a pyrrole derivative, an acrylonitrile, a methacrylonitrile, a crotonitrile, an acrylamide, a methacrylamide or a crotonamide, preferably an acrylonitrile, methacrylonitrile or crotonitrile, most preferably an acrylonitrile.

Preferably, the flakes are heated in step (f) initially to from 100° C. to 300° C. in an oxygen-containing atmosphere and then to from 200 to 600° C. in an inert gas atmosphere.

The present invention therefore relates also to pigments based on the synthetic mica flakes according to the invention comprising over the entire surface of the silicon oxide, or titanium oxide coated synthetic mica flakes a layer consisting of from 50 to 95% by weight carbon, from 5 to 25% by weight nitrogen and from 0 to 25% by weight of the elements hydrogen, oxygen and/or sulfur, the percentage by weight data relating to the total weight of the layer (PAN).

The thickness of the nitrogen-doped carbon layer is generally from 10 to 150 nm, preferably from 30 to 70 nm. In said embodiment preferred pigments have the following layer structure:

Synthetic hectorite substrate/TiO₂/PAN, synthetic hectorite substrate/TiO₂/PAN/TiO₂, synthetic hectorite substrate/TiO₂/PAN/SiO₂/PAN.

In an especially preferred embodiment, the interference pigments on the basis of the synthetic mica substrate comprise a layer of a dielectric material having a “high” refractive index, that is to say a refractive index greater than about 1.65, preferably greater than about 2.0, most preferred greater than about 2.2, which is applied to the entire surface of the synthetic mica substrate. Examples of such a dielectric material are zinc sulfide (ZnS), zinc oxide (ZnO), zirconium oxide (ZrO₂), titanium dioxide (TiO₂), carbon, indium oxide (In₂O₃), indium tin oxide (ITO), tantalum pentoxide (Ta₂O₅), chromium oxide (Cr₂O₃), cerium oxide (CeO₂), yttrium oxide (Y₂O₃), europium oxide (Eu₂O₃), iron oxides such as iron(II)/iron(III) oxide (Fe₃O₄) and iron(III) oxide (Fe₂O₃), hafnium nitride (HfN), hafnium carbide (HfC), hafnium oxide (HfO₂), lanthanum oxide (La₂O₃), magnesium oxide (MgO), neodymium oxide (Nd₂O₃), praseodymium oxide (Pr₆O₁₁), samarium oxide (Sm₂O₃), antimony trioxide (Sb₂O₃), silicon monoxides (SiO), selenium trioxide (Se₂O₃), tin oxide (SnO₂), tungsten trioxide (WO₃), or combinations thereof. The dielectric material is preferably a metal oxide. It being possible for the metal oxide to be a single oxide or a mixture of oxides, with or without absorbing properties, for example, TiO₂, ZrO₂, Fe₂O₃, Fe₃O₄, Cr₂O₃ or ZnO, with TiO₂ being especially preferred.

It is possible to obtain pigments that are more intense in colour and more transparent by applying, on top of the TiO₂ layer, a metal oxide of low refractive index, such as SiO₂, Al₂O₃, AlOOH, B₂O₃ or a mixture thereof, preferably SiO₂, and optionally applying a further TiO₂ layer on top of the latter layer (EP-A-892832, EP-A-753545, WO93/08237, WO98/53011, WO9812266, WO9838254, WO99/20695, WO00/42111, and EP-A-1213330). Nonlimiting examples of suitable low index dielectric materials that can be used include silicon dioxide (SiO₂), aluminum oxide (Al₂O₃), and metal fluorides such as magnesium fluoride (MgF₂), aluminum fluoride (AlF₃), cerium fluoride (CeF₃), lanthanum fluoride (LaF₃), sodium aluminum fluorides (e.g., Na₃AlF₆ or Na₅Al₃F₁₄), neodymium fluoride (NdF₃), samarium fluoride (SmF₃), barium fluoride (BaF₂), calcium fluoride (CaF₂), lithium fluoride (LiF), combinations thereof, or any other low index material having an index of refraction of about 1.65 or less. For example, organic monomers and polymers can be utilized as low index materials, including dienes or alkenes such as acrylates (e.g., methacrylate), polymers of perfluoroalkenes, polytetrafluoroethylene (TEFLON), polymers of fluorinated ethylene propylene (FEP), parylene, p-xylene, combinations thereof, and the like. Additionally, the foregoing materials include evaporated, condensed and cross-linked transparent acrylate layers, which may be deposited by methods described in U.S. Pat. No. 5,877,895, the disclosure of which is incorporated herein by reference.

Accordingly, preferred interference pigments comprise besides (a) a metal oxide of high refractive index in addition (b) a metal oxide of low refractive index, wherein the difference of the refractive indices is at least 0.1.

Pigments on the basis of the synthetic mica substrates, which have been coated by a wet chemical method, in the indicated order are particularly preferred:

TiO₂, (SnO₂)TiO₂ (substrate: synthetic mica; layer: (SnO₂)TiO₂, preferably in the rutile modification), titanium suboxide, TiO₂/titanium suboxide, Fe₂O₃, Fe₃O₄, TiFe₂O₅, FeTiO₃, Cr₂O₃, ZrO₂, Sn(Sb)O₂, BiOCl, Al₂O₃, Ce₂S₃, MoS₂, Fe₂O₃.TiO₂ (substrate: synthetic mica; mixed layer of Fe₂O₃ and TiO₂), TiO₂/Fe₂O₃ (substrate: synthetic mica; first layer: TiO₂; second layer: Fe₂O₃), TiO₂/Berlin blau, TiO₂/Cr₂O₃, or TiO₂/FeTiO₃. In general the layer thickness ranges from 1 to 1000 nm, preferably from 1 to 300 nm.

In another particularly preferred embodiment the present invention relates to interference pigments containing at least three alternating layers of high and low refractive index, such as, for example, TiO₂/SiO₂/TiO₂, (SnO₂)TiO₂/SiO₂/TiO₂, TiO₂/SiO₂/TiO₂/SiO₂/TiO₂, Fe₂O₃/SiO₂/TiO₂, or TiO₂/SiO₂/Fe₂O₃.

Preferably the layer structure is as follows:
(a) a coating having a refractive index>1.65,
(b) a coating having a refractive index≦1.65,
(c) a coating having a refractive index>1.65, and
(d) optionally an outer protective layer.

The thickness of the individual layers of high and low refractive index on the base substrate is essential for the optical properties of the pigment. The thickness of the individual layers, especially metal oxide layers, depends on the field of use and is generally 10 to 1000 nm, preferably 15 to 800 nm, in particular 20 to 600 nm.

The thickness of layer (A) is 10 to 550 nm, preferably 15 to 400 nm and, in particular, 20 to 350 nm. The thickness of layer (B) is 10 to 1000 nm, preferably 20 to 800 nm and, in particular, 30 to 600 nm. The thickness of layer (C) is 10 to 550 nm, preferably 15 to 400 nm and, in particular, 20 to 350 nm.

Particularly suitable materials for layer (A) are metal oxides, metal sulfides, or metal oxide mixtures, such as TiO₂, Fe₂O₃, TiFe₂O₅, Fe₃O₄, BiOCl, CoO, Co₃O₄, Cr₂O₃, VO₂, V₂O₃, Sn(Sb)O₂, SnO₂, ZrO₂, iron titanates, iron oxide hydrates, titanium suboxides (reduced titanium species having oxidation states from 2 to <4), bismuth vanadate, cobalt aluminate, and also mixtures or mixed phases of these compounds with one another or with other metal oxides. Metal sulfide coatings are preferably selected from sulfides of tin, silver, lanthanum, rare earth metals, preferably cerium, chromium, molybdenum, tungsten, iron, cobalt and/or nickel.

Particularly suitable materials for layer (B) are metal oxides or the corresponding oxide hydrates, such as SiO₂, MgF₂, Al₂O₃, AlOOH, B₂O₃ or a mixture thereof, preferably SiO₂.

Particularly suitable materials for layer (C) are colorless or colored metal oxides, such as TiO₂, Fe₂O₃, TiFe₂O₅, Fe₃O₄, BiOCl, CoO, Co₃O₄, Cr₂O₃, VO₂, V₂O₃, Sn(Sb)O₂, SnO₂, ZrO₂, iron titanates, iron oxide hydrates, titanium suboxides (reduced titanium species having oxidation states from 2 to <4), bismuth vanadate, cobalt aluminate, and also mixtures or mixed phases of these compounds with one another or with other metal oxides. The TiO₂ layers can additionally contain an absorbing material, such as carbon, selectively absorbing colorants, selectively absorbing metal cations, can be coated with absorbing material, or can be partially reduced.

Interlayers of absorbing or nonabsorbing materials can be present between layers (A), (B), (C) and (D). The thickness of the interlayers is 1 to 50 nm, preferably 1 to 40 nm and, in particular, 1 to 30 nm. Such an interlayer can, for example, consist of SnO₂. It is possible to force the rutile structure to be formed by adding small amounts of SnO₂ (see, for example, WO93/08237).

In this embodiment preferred interference pigments have the following layer structure:

| | | | |
|---|---|---|---|
| Synthetic | TiO₂ | SiO₂ | TiO₂ |
| zinc | | | |
| hectorite* | | | |
| Synthetic | TiO₂ | SiO₂ | Fe₂O₃ |
| zinc | | | |
| hectorite | | | |
| Synthetic | TiO₂ | SiO₂ | TiO₂•Fe₂O₃ |
| zinc | | | |
| hectorite | | | |
| Synthetic | TiO₂ | SiO₂ | (Sn,Sb)O₂ |
| zinc | | | |
| hectorite | | | |
| Synthetic | (Sn,Sb)O₂ | SiO₂ | TiO₂ |
| zinc | | | |
| hectorite | | | |
| Synthetic | Fe₂O₃ | SiO₂ | (Sn,Sb)O₂ |
| zinc | | | |
| hectorite | | | |
| Synthetic | TiO₂•Fe₂O₃ | SiO₂ | TiO₂•Fe₂O₃ |
| zinc | | | |
| hectorite | | | |
| Synthetic | TiO₂ | SiO₂ | MoS₂ |
| zinc | | | |
| hectorite | | | |
| Synthetic | TiO₂ | SiO₂ | Cr₂O₃ |
| zinc | | | |
| hectorite | | | |
| Synthetic | Cr₂O₃ | SiO₂ | TiO₂ |
| zinc | | | |
| hectorite | | | |
| Synthetic | Fe₂O₃ | SiO₂ | TiO₂ |
| zinc | | | |
| hectorite | | | |
| Synthetic | Fe₂O₃ | SiO₂ | Fe₂O₃ |
| zinc | | | |
| hectorite | | | |
| Synthetic | Fe₂O₃ | SiO₂ | Fe₂O₃ |
| zinc | | | |
| hectorite | | | |
| Synthetic | Fe₂O₃ | Al₂O₃ | Fe₂O₃ |
| zinc | | | |
| hectorite | | | |
| Synthetic | TiO₂ | Al₂O₃ | TiO₂ |
| zinc | | | |
| hectorite | | | |
| Synthetic | Fe₂TiO₅ | SiO₂ | TiO₂ |
| zinc | | | |
| hectorite | | | |
| Synthetic | TiO₂ | SiO₂ | Fe₂TiO₅/TiO₂ |
| zinc | | | |
| hectorite | | | |
| Synthetic | TiO suboxides | SiO₂ | TiO suboxides |
| zinc | | | |
| hectorite | | | |
| Synthetic | TiO₂ | SiO₂ | TiO₂/SiO₂/TiO₂ + |
| zinc | | | Prussian Blue |
| hectorite | | | |
| Synthetic | TiO₂ | SiO₂ | TiO₂/SiO₂/TiO₂ |
| zinc | | | |
| hectorite | | | |
| Synthetic | TiO₂/SiO₂/TiO₂ | SiO₂ | TiO₂/SiO₂/TiO₂ |
| zinc | | | |
| hectorite | | | |

*synthetic Zn hectorite of a diameter of equal to 2 microns or greater.

The metal oxide layers can be applied by CVD (chemical vapour deposition) or by wet chemical coating. The metal oxide layers can be obtained by decomposition of metal carbonyls in the presence of water vapour (relatively low molecular weight metal oxides such as magnetite) or in the presence of oxygen and, where appropriate, water vapour (e.g. nickel oxide and cobalt oxide). The metal oxide layers are especially applied by means of oxidative gaseous phase decomposition of metal carbonyls (e.g. iron pentacarbonyl, chromium hexacarbonyl; EP-A-45 851), by means of hydrolytic gaseous phase decomposition of metal alcoholates (e.g. titanium and zirconium tetra-n- and -iso-propanolate; DE-A-41 40 900) or of metal halides (e.g. titanium tetrachloride; EP-A-338 428), by means of oxidative decomposition of organyl tin compounds (especially alkyl tin compounds such as tetrabutyltin and tetramethyltin; DE-A-44 03 678) or by means of the gaseous phase hydrolysis of organyl silicon compounds (especially di-tert-butoxyacetoxysilane) described in EP-A-668 329, it being possible for the coating operation to be carried out in a fluidised-bed reactor (EP-A-045 851 and EP-A-106 235). Al₂O₃ layers (B) can advantageously be obtained by controlled oxidation during the cooling of aluminium-coated pigments, which is otherwise carried out under inert gas (DE-A-195 16 181).

Phosphate-, chromate- and/or vanadate-containing and also phosphate- and SiO₂-containing metal oxide layers can be applied in accordance with the passivation methods described in DE-A-42 36 332 and in EP-A-678 561 by means of hydrolytic or oxidative gaseous phase decomposition of oxide-halides of the metals (e.g. CrO₂Cl₂, VOCl₃), especially of phosphorus oxyhalides (e.g. POCl₃), phosphoric and phosphorous acid esters (e.g. di- and tri-methyl and di- and tri-ethyl phosphite) and of amino-group-containing organyl silicon compounds (e.g. 3-aminopropyl-triethoxy- and -trimethoxy-silane).

Layers of oxides of the metals zirconium, titanium, iron and zinc, oxide hydrates of those metals, iron titanates, titanium suboxides or mixtures thereof are preferably applied by precipitation by a wet chemical method, it being possible, where appropriate, for the metal oxides to be reduced. In the case of the wet chemical coating, the wet chemical coating methods developed for the production of pearlescent pigments may be used; these are described, for example, in DE-A-14 67 468, DE-A-19 59 988, DE-A-20 09 566, DE-A-22 14 545, DE-A-22 15 191, DE-A-22 44 298, DE-A-23 13 331, DE-A-25 22 572, DE-A-31 37 808, DE-A-31 37 809, DE-A-31 51 343, DE-A-31 51 354, DE-A-31 51 355, DE-A-32 11 602 and DE-A-32 35 017, DE 195 99 88, WO 93/08237, WO 98/53001 and WO03/6558.

The metal oxide of high refractive index is preferably TiO₂ and/or iron oxide, and the metal oxide of low refractive index is preferably SiO₂. Layers of TiO₂ can be in the rutile or anastase modification, wherein the rutile modification is preferred. TiO₂ layers can also be reduced by known means, for example ammonia, hydrogen, hydrocarbon vapor or mixtures thereof, or metal powders, as described in EP-A-735,114, DE-A-3433657, DE-A-4125134, EP-A-332071, EP-A-707,050, WO93/19131, or WO06/131472.

For the purpose of coating, the substrate particles are suspended in water and one or more hydrolysable metal salts are added at a pH suitable for the hydrolysis, which is so selected that the metal oxides or metal oxide hydrates are precipitated directly onto the particles without subsidiary precipitation occurring. The pH is usually kept constant by simultaneously metering in a base. The pigments are then separated off, washed, dried and, where appropriate, calcinated, it being possible to optimise the calcinating temperature with respect to the coating in question. If desired, after individual coatings have been applied, the pigments can be separated off, dried and, where appropriate, calcinated, and then again re-suspended for the purpose of precipitating further layers.

The metal oxide layers are also obtainable, for example, in analogy to a method described in DE-A-195 01 307, by producing the metal oxide layer by controlled hydrolysis of one or more metal acid esters, where appropriate in the presence of an organic solvent and a basic catalyst, by means of a sol-gel process. Suitable basic catalysts are, for example, amines, such as triethylamine, ethylenediamine, tributylamine, dimethylethanolamine and methoxy-propylamine. The organic solvent is a water-miscible organic solvent such as a C₁₋₄alcohol, especially isopropanol.

Suitable metal acid esters are selected from alkyl and aryl alcoholates, carboxylates, and carboxyl-radical- or alkyl-radical- or aryl-radical-substituted alkyl alcoholates or carboxylates of vanadium, titanium, zirconium, silicon, aluminium and boron. The use of triisopropyl aluminate, tetraisopropyl titanate, tetraisopropyl zirconate, tetraethyl orthosilicate and triethyl borate is preferred. In addition, acetylacetonates and acetoacetylacetonates of the afore-mentioned metals may be used. Preferred examples of that type of metal acid ester are zirconium acetylacetonate, aluminium acetylacetonate, titanium acetylacetonate and diisobutyloleyl acetoacetylaluminate or diisopropyloleyl acetoacetylacetonate and mixtures of metal acid esters, for example Dynasil® (Hüls), a mixed aluminium/silicon metal acid ester.

As a metal oxide having a high refractive index, titanium dioxide is preferably used, the method described in U.S. Pat. No. 3,553,001 being used, in accordance with an embodiment of the present invention, for application of the titanium dioxide layers.

An aqueous titanium salt solution is slowly added to a suspension of the material being coated, which suspension has been heated to about 50-100° C., especially 70-80° C., and a substantially constant pH value of about from 0.5 to 5, especially about from 1.2 to 2.5, is maintained by simultaneously metering in a base such as, for example, aqueous ammonia solution or aqueous alkali metal hydroxide solution. As soon as the desired layer thickness of precipitated TiO₂ has been achieved, the addition of titanium salt solution and base is stopped. Addition of a precursor for Al₂O₃ or MgO in the starting solutions is a way for improving the morphology of the TiO₂ layer.

This method, also referred to as the “titration method”, is distinguished by the fact that an excess of titanium salt is avoided. That is achieved by feeding in for hydrolysis, per unit time, only that amount which is necessary for even coating with the hydrated TiO₂ and which can be taken up per unit time by the available surface of the particles being coated. In principle, the anatase form of TiO₂ forms on the surface of the starting pigment. By adding small amounts of SnO₂, however, it is possible to force the rutile structure to be formed. For example, as described in WO 93/08237, tin dioxide can be deposited before titanium dioxide precipitation and the product coated with titanium dioxide can be calcined at from 800 to 900° C.

In an especially preferred embodiment of the present invention the synthetic mica flakes are mixed with distilled water in a closed reactor and heated at about 90° C. The pH is set to about 1.8 to 2.2 and a preparation comprising TiOCl₂, HCl, glycine and distilled water is added slowly while keeping the pH constant (1.8 to 2.2) by continuous addition of 1M NaOH solution. Reference is made to European patent application PCT/EP2008/051910. By adding an amino acid, such as glycine, during the deposition of the TiO₂ it is possible to improve the quality of the TiO₂ coating to be formed. Advantageously, a preparation comprising TiOCl₂, HCl, and glycine and distilled water is added to the substrate flakes in water.

The TiO₂ can optionally be reduced by usual procedures: U.S. Pat. No. 4,948,631 (NH₃, 750-850° C.), WO93/19131 (H₂, >900° C.) or DE-A-19843014 (solid reduction agent, such as, for example, silicon, >600° C.).

Where appropriate, an SiO₂ (protective) layer can be applied on top of the titanium dioxide layer, for which the following method may be used: A soda waterglass solution is metered into a suspension of the material being coated, which suspension has been heated to about 50-100° C., especially 70-80° C. The pH is maintained at from 4 to 10, preferably from 6.5 to 8.5, by simultaneously adding 10% hydrochloric acid. After addition of the waterglass solution, stirring is carried out for 30 minutes.

It is possible to obtain pigments that are more intense in colour and more transparent by applying, on top of the TiO₂ layer, a metal oxide of “low” refractive index, that is to say a refractive index smaller than about 1.65, such as SiO₂, Al₂O₃, AlOOH, B₂O₃ or a mixture thereof, preferably SiO₂, and applying a further Fe₂O₃ and/or TiO₂ layer on top of the latter layer. Such multi-coated interference pigments comprising a synthetic mica substrate and alternating metal oxide layers of with high and low refractive index can be prepared in analogy to the processes described in WO98/53011 and WO99/20695.

It is, in addition, possible to modify the powder colour of the pigment by applying further layers such as, for example, coloured metal oxides or Berlin Blue, compounds of transition metals, e.g. Fe, Cu, Ni, Co, Cr, or organic compounds such as dyes or colour lakes.

In addition, the pigment according to the invention can also be coated with poorly soluble, firmly adhering, inorganic or organic colourants. Preference is given to the use of colour lakes and, especially, aluminium colour lakes. For that purpose an aluminium hydroxide layer is precipitated, which is, in a second step, laked by using a colour lake (DE-A-24 29 762 and DE-A-29 28 287).

Furthermore, the pigment according to the invention may also have an additional coating with complex salt pigments, especially cyanoferrate complexes (EP-A-141 173 and DE-A-23 13 332).

To enhance the weather and light stability the (multilayer) synthetic mica flakes can be, depending on the field of application, subjected to a surface treatment. Useful surface treatments are, for example, described in DE-A-2215191, DE-A-3151354, DE-A-3235017, DE-A-3334598, DE-A-4030727, EP-A-649886, WO97/29059, WO99/57204, and U.S. Pat. No. 5,759,255. Said surface treatment might also facilitate the handling of the pigment, especially its incorporation into various application media.

In a preferred embodiment of the present invention is directed to pigments which contain a core of synthetic mica and comprise a mixed layer of Al₂O₃/TiO₂. The mixed layer can contain up to 20 mol % Al₂O₃. The mixed layer of Al₂O₃/TiO₂ is obtained by slowly adding an aqueous aluminum and titanium salt solution to a suspension of the material being coated, which suspension has been heated to about 50-100° C., especially 70-80° C., and maintaining a substantially constant pH value of about from 0.5 to 5, especially about from 1.2 to 2.5, by simultaneously metering in a base such as, for example, aqueous ammonia solution or aqueous alkali metal hydroxide solution. As soon as the desired layer thickness of precipitated Al₂O₃/TiO₂ has been achieved, the addition of titanium and aluminum salt solution and base is stopped.

The thickness of the mixed layer of Al₂O₃/TiO₂ is in general in the range of 20 to 200 nm, especially 50 to 150 nm. Preferably the pigments comprise a TiO₂ layer on top of the mixed layer of Al₂O₃/TiO₂ having a thickness of 1 to 50 nm, especially 10 to 20 nm. By varying the thickness of the mixed layer of Al₂O₃/TiO₂ the flop of the pigments can be enhanced and controlled as desired.

In another preferred embodiment of the present invention is directed to pigments which contain a core of the synthetic hectorite of diameter 2 or greater and consist of subsequent layers of TiO₂/SnO₂/TiO₂, wherein the TiO₂ layer next to the synthetic mica substrate has a thickness of 1 to 20 nm and is preferably prepared by using titanium alcoholates, especially tetraisopropyl titanate.

The platelet-like substrate (core) of the pigments of the present invention consists of synthetic zinc hectorite of diameter of 2 or greater.

Metallic or non-metallic, inorganic platelet-shaped particles or pigments are effect pigments, (especially metal effect pigments or interference pigments), that is to say, pigments that, besides imparting colour to an application medium, impart additional properties, for example angle dependency of the colour (flop), lustre (not surface gloss) or texture. On metal effect pigments, substantially oriented reflection occurs at directionally oriented pigment particles. In the case of interference pigments, the colour-imparting effect is due to the phenomenon of interference of light in thin, highly refractive layers.

The (effect) pigments according to the invention can be used for all customary purposes, for example for colouring polymers in the mass, coatings (including effect finishes, including those for the automotive sector) and printing inks (including offset printing, intaglio printing, bronzing and flexographic printing), and also, for example, for applications in cosmetics, in ink-jet printing, for dyeing textiles, glazes for ceramics and glass as well as laser marking of papers and plastics. Such applications are known from reference works, for example “Industrielle Organische Pigmente” (W. Herbst and K. Hunger, VCH Verlagsgesellschaft mbH, Weinheim/New York, 2nd, completely revised edition, 1995).

When the pigments according to the invention are interference pigments (effect pigments), they may be goniochromatic and result in brilliant, highly saturated (lustrous) colours. They are accordingly very especially suitable for combination with conventional, transparent pigments, for example organic pigments such as, for example, diketopyrrolopyrroles, quinacridones, dioxazines, perylenes, isoindolinones etc., it being possible for the transparent pigment to have a similar colour to the effect pigment. Especially interesting combination effects are obtained, however, in analogy to, for example, EP-A-388 932 or EP-A-402 943, when the colour of the transparent pigment and that of the effect pigment are complementary.

The pigments according to the invention can be used with excellent results for pigmenting high molecular weight organic material.

The high molecular weight organic material for the pigmenting of which the pigments or pigment compositions according to the invention may be used may be of natural or synthetic origin. High molecular weight organic materials usually have average weight average molecular weights of about from 10³ to 10⁸ g/mol or even more. They may be, for example, natural resins, drying oils, rubber or casein, or natural substances derived therefrom, such as chlorinated rubber, oil-modified alkyd resins, viscose, cellulose ethers or esters, such as ethylcellulose, cellulose acetate, cellulose propionate, cellulose acetobutyrate or nitrocellulose, but especially totally synthetic organic polymers (thermosetting plastics and thermoplastics), as are obtained by polymerisation, polycondensation or polyaddition. From the class of the polymerisation resins there may be mentioned, especially, polyolefins, such as polyethylene, polypropylene or polyisobutylene, and also substituted polyolefins, such as polymerisation products of vinyl chloride, vinyl acetate, styrene, acrylonitrile, acrylic acid esters, methacrylic acid esters or butadiene, and also copolymerisation products of the said monomers, such as especially ABS or EVA.

From the series of the polyaddition resins and polycondensation resins there may be mentioned, for example, condensation products of formaldehyde with phenols, so-called phenoplasts, and condensation products of formaldehyde with urea, thiourea or melamine, so-called aminoplasts, and the polyesters used as surface-coating resins, either saturated, such as alkyd resins, or unsaturated, such as maleate resins; also linear polyesters and polyamides, polyurethanes or silicones.

The said high molecular weight compounds may be present singly or in mixtures, in the form of plastic masses or melts. They may also be present in the form of their monomers or in the polymerised state in dissolved form as film-formers or binders for coatings or printing inks, such as, for example, boiled linseed oil, nitrocellulose, alkyd resins, melamine resins and urea-formaldehyde resins or acrylic resins.

Depending on the intended purpose, it has proved advantageous to use the effect pigments or effect pigment compositions according to the invention as toners or in the form of preparations. Depending on the conditioning method or intended application, it may be advantageous to add certain amounts of texture-improving agents to the effect pigment before or after the conditioning process, provided that this has no adverse effect on use of the effect pigments for colouring high molecular weight organic materials, especially polyethylene. Suitable agents are, especially, fatty acids containing at least 18 carbon atoms, for example stearic or behenic acid, or amides or metal salts thereof, especially magnesium salts, and also plasticisers, waxes, resin acids, such as abietic acid, rosin soap, alkylphenols or aliphatic alcohols, such as stearyl alcohol, or aliphatic 1,2-dihydroxy compounds containing from 8 to 22 carbon atoms, such as 1,2-dodecanediol, and also modified colophonium maleate resins or fumaric acid colophonium resins. The texture-improving agents are added in amounts of preferably from 0.1 to 30% by weight, especially from 2 to 15% by weight, based on the end product.

The (effect) pigments according to the invention can be added in any tinctorially effective amount to the high molecular weight organic material being pigmented. A pigmented substance composition comprising a high molecular weight organic material and from 0.01 to 80% by weight, preferably from 0.1 to 30% by weight, based on the high molecular weight organic material, of an pigment according to the invention is advantageous. Concentrations of from 1 to 20% by weight, especially of about 10% by weight, can often be used in practice.

High concentrations, for example those above 30% by weight, are usually in the form of concentrates (“masterbatches”) which can be used as colorants for producing pigmented materials having a relatively low pigment content, the pigments according to the invention having an extraordinarily low viscosity in customary formulations so that they can still be processed well.

For the purpose of pigmenting organic materials, the effect pigments according to the invention may be used singly. It is, however, also possible, in order to achieve different hues or colour effects, to add any desired amounts of other colour-imparting constituents, such as white, coloured, black or effect pigments, to the high molecular weight organic substances in addition to the effect pigments according to the invention. When coloured pigments are used in admixture with the effect pigments according to the invention, the total amount is preferably from 0.1 to 10% by weight, based on the high molecular weight organic material. Especially high goniochromicity is provided by the preferred combination of an effect pigment according to the invention with a coloured pigment of another colour, especially of a complementary colour, with colorations made using the effect pigment and colorations made using the coloured pigment having, at a measurement angle of 10°, a difference in hue (ΔH*) of from 20 to 340, especially from 150 to 210.

Preferably, the effect pigments according to the invention are combined with transparent coloured pigments, it being possible for the transparent coloured pigments to be present either in the same medium as the effect pigments according to the invention or in a neighbouring medium. An example of an arrangement in which the effect pigment and the coloured pigment are advantageously present in neighbouring media is a multi-layer effect coating.

The pigmenting of high molecular weight organic substances with the pigments according to the invention is carried out, for example, by admixing such a pigment, where appropriate in the form of a masterbatch, with the substrates using roll mills or mixing or grinding apparatuses. The pigmented material is then brought into the desired final form using methods known per se, such as calendering, compression moulding, extrusion, coating, pouring or injection moulding. Any additives customary in the plastics industry, such as plasticisers, fillers or stabilisers, can be added to the polymer, in customary amounts, before or after incorporation of the pigment. In particular, in order to produce non-rigid shaped articles or to reduce their brittleness, it is desirable to add plasticisers, for example esters of phosphoric acid, phthalic acid or sebacic acid, to the high molecular weight compounds prior to shaping.

For pigmenting coatings and printing inks, the high molecular weight organic materials and the effect pigments according to the invention, where appropriate together with customary additives such as, for example, fillers, other pigments, siccatives or plasticisers, are finely dispersed or dissolved in the same organic solvent or solvent mixture, it being possible for the individual components to be dissolved or dispersed separately or for a number of components to be dissolved or dispersed together, and only thereafter for all the components to be brought together.

Dispersing an effect pigment according to the invention in the high molecular weight organic material being pigmented, and processing a pigment composition according to the invention, are preferably carried out subject to conditions under which only relatively weak shear forces occur so that the effect pigment is not broken up into smaller portions.

Plastics comprising the pigment of the invention in amounts of 0.1 to 50% by weight, in particular 0.5 to 7% by weight. In the coating sector, the pigments of the invention are employed in amounts of 0.1 to 10% by weight. In the pigmentation of binder systems, for example for paints and printing inks for intaglio, offset or screen printing, the pigment is incorporated into the printing ink in amounts of 0.1 to 50% by weight, preferably 5 to 30% by weight and in particular 8 to 15% by weight.

The colorations obtained, for example in plastics, coatings or printing inks, especially in coatings or printing inks, more especially in coatings, may be distinguished by excellent properties, especially by extremely high saturation, outstanding fastness properties, high color purity and high goniochromaticity.

When the high molecular weight material being pigmented is a coating, it is especially a speciality coating, very especially an automotive finish.

The effect pigments according to the invention are also suitable for making-up the lips or the skin and for colouring the hair or the nails.

The invention accordingly relates also to a cosmetic preparation or formulation comprising from 0.0001 to 90% by weight of a pigment, especially an effect pigment, according to the invention and from 10 to 99.9999% of a cosmetically suitable carrier material, based on the total weight of the cosmetic preparation or formulation.

Such cosmetic preparations or formulations are, for example, lipsticks, blushers, foundations, nail varnishes and hair shampoos.

The pigments may be used singly or in the form of mixtures. It is, in addition, possible to use pigments according to the invention together with other pigments and/or colorants, for example in combinations as described hereinbefore or as known in cosmetic preparations.

The cosmetic preparations and formulations according to the invention preferably contain the pigment according to the invention in an amount from 0.005 to 50% by weight, based on the total weight of the preparation.

Suitable carrier materials for the cosmetic preparations and formulations according to the invention include the customary materials used in such compositions.

The cosmetic preparations and formulations according to the invention may be in the form of, for example, sticks, ointments, creams, emulsions, suspensions, dispersions, powders or solutions. They are, for example, lipsticks, mascara preparations, blushers, eye-shadows, foundations, eyeliners, powder or nail varnishes.

If the preparations are in the form of sticks, for example lipsticks, eye-shadows, blushers or foundations, the preparations consist for a considerable part of fatty components, which may consist of one or more waxes, for example ozokerite, lanolin, lanolin alcohol, hydrogenated lanolin, acetylated lanolin, lanolin wax, beeswax, candelilla wax, microcrystalline wax, carnauba wax, cetyl alcohol, stearyl alcohol, cocoa butter, lanolin fatty acids, petrolatum, petroleum jelly, mono-, di- or tri-glycerides or fatty esters thereof that are solid at 25° C., silicone waxes, such as methyloctadecane-oxypolysiloxane and poly(dimethylsiloxy)stearoxysiloxane, stearic acid monoethanolamine, colophane and derivatives thereof, such as glycol abietates and glycerol abietates, hydrogenated oils that are solid at 25° C., sugar glycerides and oleates, myristates, lanolates, stearates and dihydroxystearates of calcium, magnesium, zirconium and aluminium.

The fatty component may also consist of a mixture of at least one wax and at least one oil, in which case the following oils, for example, are suitable: paraffin oil, purcelline oil, perhydrosqualene, sweet almond oil, avocado oil, calophyllum oil, castor oil, sesame oil, jojoba oil, mineral oils having a boiling point of about from 310 to 410° C., silicone oils, such as dimethylpolysiloxane, linoleyl alcohol, linolenyl alcohol, oleyl alcohol, cereal grain oils, such as wheatgerm oil, isopropyl lanolate, isopropyl palmitate, isopropyl myristate, butyl myristate, cetyl myristate, hexadecyl stearate, butyl stearate, decyl oleate, acetyl glycerides, octanoates and decanoates of alcohols and polyalcohols, for example of glycol and glycerol, ricinoleates of alcohols and polyalcohols, for example of cetyl alcohol, isostearyl alcohol, isocetyl lanolate, isopropyl adipate, hexyl laurate and octyl dodecanol.

The fatty components in such preparations in the form of sticks may generally constitute up to 99.91% by weight of the total weight of the preparation.

The cosmetic preparations and formulations according to the invention may additionally comprise further constituents, such as, for example, glycols, polyethylene glycols, polypropylene glycols, monoalkanolamides, non-coloured polymeric, inorganic or organic fillers, preservatives, UV filters or other adjuvants and additives customary in cosmetics, for example a natural or synthetic or partially synthetic di- or tri-glyceride, a mineral oil, a silicone oil, a wax, a fatty alcohol, a Guerbet alcohol or ester thereof, a lipophilic functional cosmetic active ingredient, including sun-protection filters, or a mixture of such substances.

A lipophilic functional cosmetic active ingredient suitable for skin cosmetics, an active ingredient composition or an active ingredient extract is an ingredient or a mixture of ingredients that is approved for dermal or topical application. The following may be mentioned by way of example:
active ingredients having a cleansing action on the skin surface and the hair; these include all substances that serve to cleanse the skin, such as oils, soaps, synthetic detergents and solid substances;
active ingredients having a deodorising and perspiration-inhibiting action: they include antiperspirants based on aluminium salts or zinc salts, deodorants comprising bactericidal or bacteriostatic deodorising substances, for example triclosan, hexachlorophene, alcohols and cationic substances, such as, for example, quaternary ammonium salts, and odour absorbers, for example ®Grillocin (combination of zinc ricinoleate and various additives) or triethyl citrate (optionally in combination with an antioxidant, such as, for example, butyl hydroxytoluene) or ion-exchange resins;
active ingredients that offer protection against sunlight (UV filters): suitable active ingredients are filter substances (sunscreens) that are able to absorb UV radiation from sunlight and convert it into heat; depending on the desired action, the following light-protection agents are preferred: light-protection agents that selectively absorb sunburn-causing high-energy UV radiation in the range of approximately from 280 to 315 nm (UV-B absorbers) and transmit the longer-wavelength range of, for example, from 315 to 400 nm (UV-A range), as well as light-protection agents that absorb only the longer-wavelength radiation of the UV-A range of from 315 to 400 nm (UV-A absorbers);
suitable light-protection agents are, for example, organic UV absorbers from the class of the p-aminobenzoic acid derivatives, salicylic acid derivatives, benzophenone derivatives, dibenzoylmethane derivatives, diphenyl acrylate derivatives, benzofuran derivatives, polymeric UV absorbers comprising one or more organosilicon radicals, cinnamic acid derivatives, camphor derivatives, trianilino-s-triazine derivatives, phenyl-benzimidazolesulfonic acid and salts thereof, menthyl anthranilates, benzotriazole derivatives, and/or an inorganic micropigment selected from aluminium oxide- or silicon dioxide-coated TiO₂, zinc oxide or mica;
active ingredients against insects (repellents) are agents that are intended to prevent insects from touching the skin and becoming active there; they drive insects away and evaporate slowly; the most frequently used repellent is diethyl toluamide (DEET); other common repellents will be found, for example, in “Pflegekosmetik” (W. Raab and U. Kindl, Gustav-Fischer-Verlag Stuttgart/New York, 1991) on page 161;
active ingredients for protection against chemical and mechanical influences: these include all substances that form a barrier between the skin and external harmful substances, such as, for example, paraffin oils, silicone oils, vegetable oils, PCL products and lanolin for protection against aqueous solutions, film-forming agents, such as sodium alginate, triethanolamine alginate, polyacrylates, polyvinyl alcohol or cellulose ethers for protection against the effect of organic solvents, or substances based on mineral oils, vegetable oils or silicone oils as “lubricants” for protection against severe mechanical stresses on the skin;
moisturising substances: the following substances, for example, are used as moisture-controlling agents (moisturisers): sodium lactate, urea, alcohols, sorbitol, glycerol, propylene glycol, collagen, elastin and hyaluronic acid;
active ingredients having a keratoplastic effect: benzoyl peroxide, retinoic acid, colloidal sulfur and resorcinol;
antimicrobial agents, such as, for example, triclosan or quaternary ammonium compounds;
oily or oil-soluble vitamins or vitamin derivatives that can be applied dermally: for example vitamin A (retinol in the form of the free acid or derivatives thereof), panthenol, pantothenic acid, folic acid, and combinations thereof, vitamin E (tocopherol), vitamin F; essential fatty acids; or niacinamide (nicotinic acid amide);
vitamin-based placenta extracts: active ingredient compositions comprising especially vitamins A, C, E, B₁, B₂, B₆, B₁₂, folic acid and biotin, amino acids and enzymes as well as compounds of the trace elements magnesium, silicon, phosphorus, calcium, manganese, iron or copper;
skin repair complexes: obtainable from inactivated and disintegrated cultures of bacteria of the bifidus group;
plants and plant extracts: for example arnica, aloe, beard lichen, ivy, stinging nettle, ginseng, henna, camomile, marigold, rosemary, sage, horsetail or thyme;
animal extracts: for example royal jelly, propolis, proteins or thymus extracts;
cosmetic oils that can be applied dermally: neutral oils of the Miglyol 812 type, apricot kernel oil, avocado oil, babassu oil, cottonseed oil, borage oil, thistle oil, groundnut oil, gamma-oryzanol, rosehip-seed oil, hemp oil, hazelnut oil, blackcurrant-seed oil, jojoba oil, cherry-stone oil, salmon oil, linseed oil, cornseed oil, macadamia nut oil, almond oil, evening primrose oil, mink oil, olive oil, pecan nut oil, peach kernel oil, pistachio nut oil, rape oil, rice-seed oil, castor oil, safflower oil, sesame oil, soybean oil, sunflower oil, tea tree oil, grapeseed oil or wheatgerm oil.

The preparations in stick form are preferably anhydrous but may in certain cases comprise a certain amount of water which, however, in general does not exceed 40% by weight, based on the total weight of the cosmetic preparation.

If the cosmetic preparations and formulations according to the invention are in the form of semi-solid products, that is to say in the form of ointments or creams, they may likewise be anhydrous or aqueous. Such preparations and formulations are, for example, mascaras, eyeliners, foundations, blushers, eye-shadows, or compositions for treating rings under the eyes.

If, on the other hand, such ointments or creams are aqueous, they are especially emulsions of the water-in-oil type or of the oil-in-water type that comprise, apart from the pigment, from 1 to 98.8% by weight of the fatty phase, from 1 to 98.8% by weight of the aqueous phase and from 0.2 to 30% by weight of an emulsifier.

Such ointments and creams may also comprise further conventional additives, such as, for example, perfumes, antioxidants, preservatives, gel-forming agents, UV filters, colorants, pigments, pearlescent agents, non-coloured polymers as well as inorganic or organic fillers. If the preparations are in the form of a powder, they consist substantially of a mineral or inorganic or organic filler such as, for example, talcum, kaolin, starch, polyethylene powder or polyamide powder, as well as adjuvants such as binders, colorants etc.

Such preparations may likewise comprise various adjuvants conventionally employed in cosmetics, such as fragrances, antioxidants, preservatives etc.

If the cosmetic preparations and formulations according to the invention are nail varnishes, they consist essentially of nitrocellulose and a natural or synthetic polymer in the form of a solution in a solvent system, it being possible for the solution to comprise other adjuvants, for example pearlescent agents.

In that embodiment, the coloured polymer is present in an amount of approximately from 0.1 to 5% by weight.

The cosmetic preparations and formulations according to the invention may also be used for colouring the hair, in which case they are used in the form of shampoos, creams or gels that are composed of the base substances conventionally employed in the cosmetics industry and a pigment according to the invention.

The cosmetic preparations and formulations according to the invention are prepared in conventional manner, for example by mixing or stirring the components together, optionally with heating so that the mixtures melt.

Thus the present application envisions cosmetics, coatings, inks, paints, and plastic composition containing the effect pigment formed from a coated hectorite of diameter of 2 microns or greater.

Barrier Applications of the Hydrothermally Produced Zinc Hectorite

The synthetically derived zinc hectorite platelets produced via the hydrothermal process above may be used to effect water vapor and oxygen barriers when present in paper coatings, coatings on packaging films or melt blended in films or containers used in packaging.

The platelets formed by the presently disclosed process may be used to form layered structures on or in such substrates such as paper, plastic packaging or as component within a coating. The layered structures of mica materials, for example, may be used to provide a barrier packaging film with a low moisture vapor transmission rate (MVTR), CO₂, and/or a low oxygen transmission rate (OTR).

It is well known to use layered silicates to improve the flame retardant properties of flammable substrates. For example the zinc hectorite platelet formed by the present hydrothermal process, may be used in polymeric composites for improving the flame retardant properties of the composite by increasing the barrier properties of the composite, and increased char formation upon ignition of the composite.

### EXAMPLES

### Apparent Density Determination Method

Equipment: Scott Volumeter equipped with a 16 or 40 mesh screen, metal funnel and baffle box. 1 inch³ density cup with capacity of 1.000±0.002 in³
Final powder to be tested should be free flowing and not contain lumps. The sample is poured onto the screen and the powder flows down through the baffle box and into the cup until the cup is completely filled to overflowing on all sides of the cup. Then without disturbing the cup rotate the baffle box and funnel out of the way. Remove excess powder by scoring the top of the cup diagonally with a spatual. Tap cup gently to settle powder and weigh.

### Example 1

### Synthesis of Zinc Hectorite (LiₓZn₃₋ₓLiₓSi₄O₁₀(OH)₂ without habit modifier

The starting reagents are colloidal silica, Zinc sulfate heptahyrate, and lithium hydroxide. A 2M LiOH solution is added to zinc sulfate and the contents are swirled to mix. Water is added and the reaction mixture is transferred to a Parr reactor. The colloidal silica is added forming an aqueous gel. The Parr reactor is sealed and placed in an oven at 200° C. for 24-72 hours. Upon cooling to room temperature, the reaction is filtered and washed with D.I. water to yield a white powder.

Gel Ratio: 6LiOH, 4SiO₂, 2ZnSO₄, 226 H₂O, 19 wt. % solids

### Example 2

### Synthesis of zinc Hectorite (LiₓZn₃₋ₓLiₓSi₄O₁₀(OH)₂ with habit modifier

The starting reagents are colloidal silica, zinc sulfate heptahydrate, and lithium hydroxide. A 2M LiOH solution is added to zinc sulfate and the contents are swirled to mix. Water is added along with tri-sodium citrate dihydrate and the reaction mixture is transferred to a Parr reactor. The colloidal silica is added forming an aqueous gel. The Parr reactor is sealed and placed in an oven at 200° C. for 24-72 hours. Upon cooling to room temperature, the reaction is filtered and washed with D.I. water to yield a white powder.
Gel Ratio: 6LiOH, 4SiO₂, 2ZnSO₄, 226 H₂O, 0.03M tri-sodium citrate dihydrate, 19 wt. % solids
Coated Zinc Hectorite with Titanium Dioxide
The zinc hectorite is coated with titanium dioxide according to known methods of the art to form an effect pigment.

1. A synthetic zinc hectorite platelet of formula (1)
Iₓ(Zn₃₋ₓ,Liₓ)Si₄O₁₀(X)₂ (1)
wherein
I is an interlayer monovalent cation selected from the group consisting of K⁺, Na⁺, Li⁺, NH⁴⁺ and mixtures thereof;
and
X is independently fluoride or hydroxide;
subscript x is a number ranging from >0 to 1 and including 1;
and Zn and Li are greater than 0;
and the synthetic zinc hectorite platelet is characterized by a diameter of =>2 microns.

2. The platelet according to claim 1, wherein the compound of formula (1) is selected from the group consisting of:
Liₓ(Zn₃₋ₓLiₓ)Si₄O₁₀(OH)₂, Naₓ(Zn₃₋ₓLiₓ)Si₄O₁₀(OH)₂, Kₓ(Zn₃₋ₓLiₓ)Si₄O₁₀(OH)₂, (NH₄)ₓ(Zn₃₋ₓLiₓ)Si₄O₁₀(OH)₂, Liₓ(Zn₃₋ₓLiₓ)Si₄O₁₀(F)₂, Naₓ(Zn₃₋ₓLiₓ)Si₄O₁₀(F)₂, Kₓ(Zn₃₋ₓLiₓ)Si₄O₁₀(F)₂, (NH₄)ₓ(Zn₃₋ₓLiₓ)Si₄O₁₀(F)₂, Liₓ(Zn₃₋ₓLiₓ)Si₄O₁₀(F,OH), Naₓ(Zn₃₋ₓLiₓ)Si₄O₁₀(F,OH),Kₓ(Zn₃₋ₓLiₓ)Si₄O₁₀(F,OH), (NH₄)ₓ(Zn₃₋ₓLiₓ)Si₄O₁₀(F,OH), (Li,K)ₓ(Zn₃₋ₓLiₓ)Si₄O₁₀(OH)₂, (Li,K)ₓ(Zn₃₋ₓLiₓ)Si₄O₁₀(F)₂, (Li,K)ₓ(Zn₃₋ₓ Liₓ)Si₄O₁₀(F,OH), (Li,Na)ₓ(Zn₃₋ₓLiₓ)Si₄O₁₀(OH)₂, (Li,Na)ₓ(Zn₃₋ₓLiₓ)Si₄O₁₀(F)₂, (Li,Na)ₓ(Zn₃₋ₓ Liₓ)Si₄O₁₀(F,OH), (Li,K)ₓ(Zn₃₋ₓLiₓ)Si₄O₁₀(OH)₂, (Li, K)ₓ(Zn₃₋ₓLiₓ)Si₄O₁₀(F)₂, (Li, K)ₓ(Zn₃₋ₓLiₓ)Si₄O₁₀(OH, F), (Li,NH₄)ₓ(Zn₃₋ₓLiₓ)Si₄O₁₀(OH)₂, (Li, NH₄)ₓ(Zn₃₋ₓLiₓ)Si₄O₁₀(F)₂ and (Li,NH₄)ₓ(Zn₃₋ₓLiₓ)Si₄O₁₀(OH, F).

3. The platelet according to claim 1, wherein the platelet is characterized by a d₅₀ ranging from =>2 to about 60 microns.

4. The platelet according to claim 1, wherein the platelet transmits
at least 92% light.

5. A process of preparing a hectorite platelet of formula (I) according to claim 1,
comprising the steps of:
forming a reaction mixture comprising
an I source selected from the group consisting of Na⁺, K⁺, NH₄⁺ and Li⁺ and mixtures thereof;
a lithium source;
a silicon source;
a zinc source;
optionally a fluoride source and/or hydroxide source;
a habit modifier selected from the group consisting of
a weak organic acid, weak inorganic acid and a sugar,
and
optionally a seed crystal of a preformed hectorite seed crystal,
hydrothermally treating said reaction mixture under basic conditions at a temperature ranging from about 125 to about 250° C.
and
a pressure ranging from about 50 to about 400 psi;
to form the synthetic hectorite platelet of formula (1);
and
optionally isolating the formed platelet.

6. A process of preparing a hectorite platelet of formula (1) according to claim 1,
comprising the steps of:
forming a reaction mixture comprising
an I source selected from the group consisting of Na⁺, K⁺, NH₄⁺ and Li⁺ and mixtures thereof;
a lithium source;
a silicon source;
a zinc source;
optionally a fluoride source and/or hydroxide source;
and
optionally a seed crystal of a preformed hectorite seed crystal,
hydrothermally treating said reaction mixture under basic conditions at a temperature ranging from about 125 to about 250° C.
and
a pressure ranging from about 50 to about 400 psi;
to form the synthetic hectorite platelet of formula (1);
and
optionally isolating the formed platelet
and the silicon source is colloidal silica.

7. The process according to claim 6, wherein the reaction mixture further comprises a habit modifier and the habit modifier is a weak organic acid, weak inorganic acid or a sugar.

8. The process according to claim 5, wherein the habit modifier is a weak organic acid or a weak inorganic acid, salt or hydrate thereof,
and the weak organic acid, salt or hydrate thereof is a compound of formula (I) 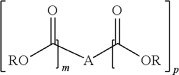
when m+p is 1:
A is branched or unbranched, substituted or unsubstituted C₁-C₁₀ alkyl, branched or unbranched, substituted or unsubstituted C₂-C₁₀ alkenyl, substituted or unsubstituted C₇-C₉ phenylalkyl or substituted or unsubstituted C₆-C₁₀ aryl,
wherein the linear or branched unsubstituted C₁-C₁₀ alkyl, the linear or branched C₂-C₁₀ alkenyl may be substituted by C(O)OH, C(O)O⁻X⁺, NH₂, halogen, OH, —C(O)H or interrupted by —O—, —NR²— or —C(O)—,
the C₇-C₉ phenylalkyl or the C₆-C₁₀ aryl may be substitution by one or more C(O)OH, C(O)O⁻X⁽⁺⁾ⁿ, NH₂, halogen, OH or —C(O)H,
R is hydrogen or RO is O⁻X⁽⁺⁾ⁿ,
R² is hydrogen or linear or branched C₁-C₁₀-alkyl one or more substituted by C(O)OH, C(O)O⁻X⁽⁺⁾ⁿ, halogen, NH₂, —C(O)— or OH; n is 1-3,
and
X⁽⁺⁾n is an organic or inorganic cation,
when m+p is two or more,
A is branched or unbranched, substituted or unsubstituted C₁-C₁₀ alkylene, branched or unbranched, substituted or unsubstituted C₂-C₁₀ alkylidene, substituted or unsubstituted C₇-C₉ alkylphenylene or C₆-C₁₀ arylene,
wherein the linear or branched C₁-C₁₀ alkylene, the linear or branched C₂-C₁₀ alkylidene may be substituted by C(O)OH, C(O)O⁻X⁺, NH₂, halogen, OH, —C(O)H and/or interrupted by —O—, —NR²— or —C(O)—,
and
the C₇-C₉ alkylphenylene or the C₆-C₁₂ arylene includes one or more substitution by NHR², OH, COOH, halogen, COO⁻X⁽⁺⁾ⁿ or —C(O)H
with R, RO, R² and X⁽⁺⁾ⁿ as defined above,
and
the weak inorganic acid is
boric acid, phosphoric acid (H₃PO₄), triphosphates, salts or hydrates thereof.

9. The process according to claim 8,
m+p is two or more,
A is branched or unbranched, substituted or unsubstituted C₁-C₈ alkylene,
substitution of the branched or unbranched C₁-C₈ alkylene includes one or more substitution by OH, COOH, COO⁻X⁽⁺⁾ⁿ as defined above, preferably the branched or unbranched C₁-C₈ alkylene is substituted by OH and
COOH or COO⁻X⁽⁺⁾ⁿ
and the weak inorganic acid is boric acid, salts or hydrates thereof.

10. The process according to claim 7, wherein the habit modifier is a weak organic acid and is selected from the group consisting of formic acid, acetic acid, acrylic acid, benzoic acid, oxalic acid, phthalic acid, isothalic acid, terephthalic acid, malonic acid, methyl malonic acid, succinic acid, lactic acid, aspartic acid, glutaric acid, adipic acid, pimelic acid, malic acid, maleic acid, tartaric acid, tartonic acid, mucic acid, gluconic acid, citric acid, acid, acetyl citric acid, suberic acid, sebacic acid, azelaic acid, 1,2,3-propanetricarboxylic acid, 1,1,3,3-propanetetracarboxylic acid, 1,1,2,2-ethane tetracarboxylic acid, 1,2,3,4-butantetetracarboxylic acid, 1,2,2,3 propanetetracarboxylic acid, 1,3,3,5 acid, ethylenediamine tetraacetic acid, ethyleneglycolbis-tetraacetic acid, diglycolic acid, ethylenediamine tetrapropionic acid, iminodiacetic acid, 1,2-propylenediaminetetraacetic acid, N-methyl, -ethyl, -propyl and -butyl iminodiacetic acid, 1,3-propylenediaminetetraacetic acid, hydroxyethylethylenediaminetriacetic acid, triethylenetetraminehexaacetic acid, diethylenetriaminepentaacetic acid, and amino acids and salts or hydrates thereof,
or
the habit modifier is a weak inorganic acid selected from the group consisting of boric acid, phosphoric acid (H₃PO₄), pyrophosphates, triphosphate and salts or hydrates thereof.

11. The process according to claim 5, wherein the habit modifier is a sugar and is selected from the group consisting of glucose, fructose, galactose, sucrose, maltose, sorbitol, lactose mannitol, inositol, xylitol, threitol, erythritol, adonitol(ribitol), arabitol(lyxitol), dulcitol(galactitol), maltitol, isomalt, ribose, xylose and mannose.

12. The process according to claim 5, wherein the amount of habit modifier added to the reaction mixture ranges from about 0.5 to about 10% mmol, based on the theoretical calculated product (hectorite).

13. The process according to claim 5, wherein the zinc source is selected from group consisting of elemental zinc Zn⁰, ZnSO₄, Zn(NO₃)₂, ZnCl₂, Zn(C₂H₃O₂)₂ (zinc acetate), ZnCO₃, Zn(CHO₂)₂ (zinc formate), ZnBr₂, zinc oxide, Zn⁰, ZnI₂ and hydrates thereof.

14. The process according to claim 5, wherein the the optional fluoride source is selected from group consisting of HF, NH₄F, NaF, K₂SiF₆, KF₋ and MgF₂ and the optional hydroxide source is selected from the group consisting of potassium hydroxide, sodium hydroxide, lithium hydroxide, ammonium hydroxide, tripropylammonium hydroxide and tetramethyl ammonium hydroxide.

15. The process according to claim 5, wherein the lithium source is selected from the group consisting of lithium acetate, lithium bromide, lithium iodide, lithium chloride, lithium fluoride, lithium carbonate, lithium citrate, lithium formate, lithium hexafluorophosphate, lithium hexafluorotitanate, lithium hydroxide, lithium oxide and hydrates thereof.

16. A pigment, comprising the zinc hectorite platelet according to claim 1.

17. A pigment comprising the hectorite platelet according to claim 1,
(a) a layer of a dielectric material; and/or
(b) a metal layer.

18. The pigment according to claim 17 wherein the pigment further comprises in addition to layer (a) having a high refractive index and/or (b) a metal layer,
an oxide layer (c) of low refractive index and a layer (d) of high refractive index, wherein the difference of the refractive indices between the high and low refractive indexes is at least 0.1.

19. The pigment according to claim 17, wherein the metal oxide of layer (a) of high refractive index is TiO₂, ZrO₂, Fe₂O₃, Fe₃O₄, Cr₂O₃, ZnO, a mixture of these oxides, an iron titanate, an iron oxide hydrate, a titanium suboxide or a mixture and/or mixed phase of these compounds.

20. A paint, ink-jet, coatings, printing ink, plastic, cosmetic, glazes for ceramics and glass containing the pigments according to claim 16.

21. A paper or plastic comprising the platelets according to claim 1.

22. Packaging comprising the paper or plastic according to claim 21.

23. A plastic according to claim 21, wherein the plastic is a film or container and platelets are melt blended in the film, container or coatings on packaging films.

24. A paper according to claim 21, wherein the platelets are present in a paper coating.

25. A barrier coating comprising the platelets according to claim 1.

26. A method of increasing the barrier properties of a paper or polymeric packaging by coating or incorporating the platelets according to claim 1

27. A method of improving the flame retardant properties of a polymeric composite by adding thereto the platelets according to claims 1.
